(19) United States
(12) Patent Application Publication
(10) Pub. No.: US 2026/0155257 A1
(43) Pub. Date: Jun. 4, 2026

(54) Identification and Use of Correlation or Absence of Correlation Between Physiological Event and User Mood

(71) Applicant: Google LLC, Mountain View, CA (US)

(72) Inventors: Julia Marie Dorothea Thomsen, London (GB); Hulya Emir-Farinas, Alameda, CA (US); Aubrey Herminia Browne, Thornton, CO (US)

(21) Appl. No.: 19/118,973

(22) PCT Filed: Dec. 7, 2022
(86) PCT No.: PCT/US2022/052131

§ 371 Date: Apr. 7, 2025

(51) Int. Cl. G16H 50/30; G16H 20/70

(52) U.S. Cl. CPC G16H 50/30; G16H 20/70

(57) ABSTRACT

According to an embodiment, a computing device includes one or more processors and one or more computer-readable media that collectively store instructions that, when executed by the processor(s), cause the computing device to perform operations. The operations can include: detecting a trigger event associated with physiological data of a user; presenting one or more mood states to the user for selection based on detecting the trigger event, the mood state(s) corresponding to at least one mood experienced by the user at a defined time associated with the trigger event; annotating the physiological data with one or more annotations indicative of the at least one mood based on selection of the mood state(s) by the user; and training a model based on the annotation(s) such that the model identifies a correlation or an absence of correlation between the trigger event and the at least one mood.

### FIELD

The present disclosure relates generally to mental and/or emotional quality assessment and alteration. More particularly, the present disclosure relates to identifying a correlation or an absence of correlation between a physiological event and a user's mood and using the correlation or absence of correlation to facilitate mental and/or emotional quality assessment and alteration.

### BACKGROUND

Many people have difficulty predicting and/or understanding their own future emotional and/or mental states. As such, it is challenging for them to know which activities will improve their short-term and/or long-term emotional and/or mental states. Additionally, when in a distressed state, many people have a tendency to remember negative information or negative correlations with certain activities, which further adds to their challenge of selecting activities that will improve their emotional and/or mental well-being.

Assessing which activities will improve a person's short-term and/or long-term emotional and/or mental state is difficult for both existing mood logging devices and wearable devices such as, for example, wrist-worn physiological monitoring devices. A problem with such existing mood logging and/or wearable devices is that they do not empower users to take control of their emotional well-being by building an awareness of their emotional and/or mental states and giving them an understanding of when and why they feel their best, so that they can create a healthy and holistic lifestyle.

### SUMMARY

Aspects and advantages of embodiments of the present disclosure will be set forth in part in the following description, or can be learned from the description, or can be learned through practice of the embodiments.

According to one example embodiment, a computing device includes one or more processors and one or more computer-readable media that collectively store instructions that, when executed by the one or more processors, cause the computing device to perform operations. The operations include detecting a trigger event associated with physiological data of a user. The operations further include presenting one or more mood states to the user for selection based at least in part on detecting the trigger event. The one or more mood states corresponding to at least one mood experienced by the user at a defined time associated with the trigger event. The operations further include annotating the physiological data with one or more annotations indicative of the at least one mood based at least in part on selection of the one or more mood states by the user. The operations further include training a model based at least in part on the one or more annotations such that the model identifies a correlation or an absence of correlation between the trigger event and the at least one mood.

According to another example embodiment, a computer-implemented method can include detecting, by a computing device operatively coupled to one or more processors, a trigger event associated with physiological data of a user. The computer-implemented method can further include presenting, by the computing device, one or more mood states to the user for selection based at least in part on detecting the trigger event. The one or more mood states corresponding to at least one mood experienced by the user at a defined time associated with the trigger event. The computer-implemented method can further include annotating, by the computing device, the physiological data with one or more annotations indicative of the at least one mood based at least in part on selection of the one or more mood states by the user. For example, annotating the physiological data includes generating or interacting with a data set (e.g., a database) to store the annotations (e.g., tags or other information indicative of the mood of the user) along with the associated physiological data. The computer-implemented method can further include training, by the computing device, a model based at least in part on the one or more annotations such that the model is capable of identifying a correlation or an absence of correlation between the trigger event and the at least one mood.

According to another example embodiment, a computing device can include one or more processors and one or more computer-readable media that collectively store instructions that, when executed by the one or more processors, cause the computing device to perform operations. The operations can include generating an annotated physiological dataset including a plurality of annotations to physiological data of a user. Each of the plurality of annotations being indicative of one or more moods experienced by the user at each of one or more defined times respectively associated with one or more defined activities performed by the user. The operations can further include identifying a correlation or an absence of correlation between a defined activity of the one or more defined activities and at least one mood of the one or more moods. The operations can further include performing one or more operations based at least in part on the correlation or the absence of correlation.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIGS. 1, 2, and 3 each illustrate a perspective view of an example, non-limiting wearable device according to one or more example embodiments of the present disclosure.

FIG. 4 illustrates a block diagram of an example, non-limiting device according to one or more example embodiments of the present disclosure.

FIGS. 5 and 6 each illustrate a diagram of an example, non-limiting user assessment management system according to one or more example embodiments of the present disclosure.

FIG. 7 illustrates an example, non-limiting physiological data graph according to one or more example embodiments of the present disclosure.

FIGS. 8 and 9 each illustrate example, non-limiting interactive user interfaces according to one or more example embodiments of the present disclosure.

FIGS. 10 and 11 each illustrate a flow diagram of an example, non-limiting computer-implemented method according to one or more example embodiments of the present disclosure.

Repeated use of reference characters and/or numerals in the present specification and/or figures is intended to represent the same or analogous features, elements, or operations of the present disclosure. Repeated description of reference characters and/or numerals that are repeated in the present specification is omitted for brevity.

### DETAILED DESCRIPTION

### Overview

As referred to herein, the terms “includes” and “including” are intended to be inclusive in a manner similar to the term “comprising.” As referenced herein, the terms “or” and “and/or” are generally intended to be inclusive, that is (i.e.), “A or B” or “A and/or B” are each intended to mean “A or B or both.” As referred to herein, the terms “first,” “second,” “third,” and so on, can be used interchangeably to distinguish one component or entity from another and are not intended to signify location, functionality, or importance of the individual components or entities. As referenced herein, the terms “couple,” “couples,” “coupled,” and/or “coupling” refer to chemical coupling (e.g., chemical bonding), communicative coupling, electrical and/or electromagnetic coupling (e.g., capacitive coupling, inductive coupling, direct and/or connected coupling, etc.), mechanical coupling, operative coupling, optical coupling, and/or physical coupling.

As referenced herein, the term “system” can refer to hardware (e.g., application specific hardware), computer logic that executes on a general-purpose processor (e.g., a central processing unit (CPU)), and/or some combination thereof. In some embodiments, a “system” described herein can be implemented in hardware, application specific circuits, firmware, and/or software controlling a general-purpose processor. In some embodiments, a “system” described herein can be implemented as program code files stored on a storage device, loaded into a memory and executed by a processor, and/or can be provided from computer program products, for example, computer-executable instructions that are stored in a tangible computer-readable storage medium (e.g., random-access memory (RAM), hard disk, optical media, magnetic media).

Example aspects of the present disclosure are directed to learning correlations or absences of correlation between trigger events associated with physiological data of a user and moods experienced by the user at defined times associated with the trigger events. More specifically, example embodiments described herein are directed to identifying a correlation or an absence of correlation between a trigger event associated with physiological data of a user and at least one mood experienced by the user at a defined time associated with the trigger event and/or using such a correlation or absence of correlation to perform one or more operations that can facilitate alteration (e.g., improvement) of the user's health quality.

According to example embodiments of the present disclosure, a computing device (e.g., a server, a client computing device, a computer, a laptop, a tablet, a smartphone, a physiological monitoring device, a wearable computing device, a wearable physiological monitoring device (e.g., a wrist-worn device, a chest strap device)) can learn a correlation or an absence of correlation between a trigger event associated with physiological data of a user and at least one mood experienced by the user at a defined time associated with the trigger event. More specifically, in at least one embodiment described herein, the computing device can identify the correlation or absence of correlation between the trigger event and the at least one mood of the user at the defined time associated with the trigger event and further use such correlation or absence of correlation to perform one or more operations that can facilitate alteration (e.g., improvement) of the user's health quality.

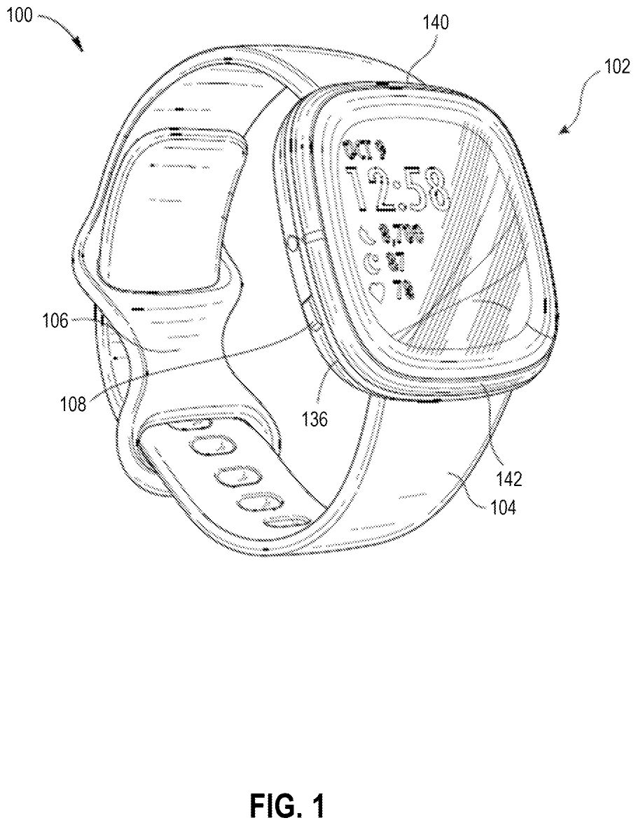

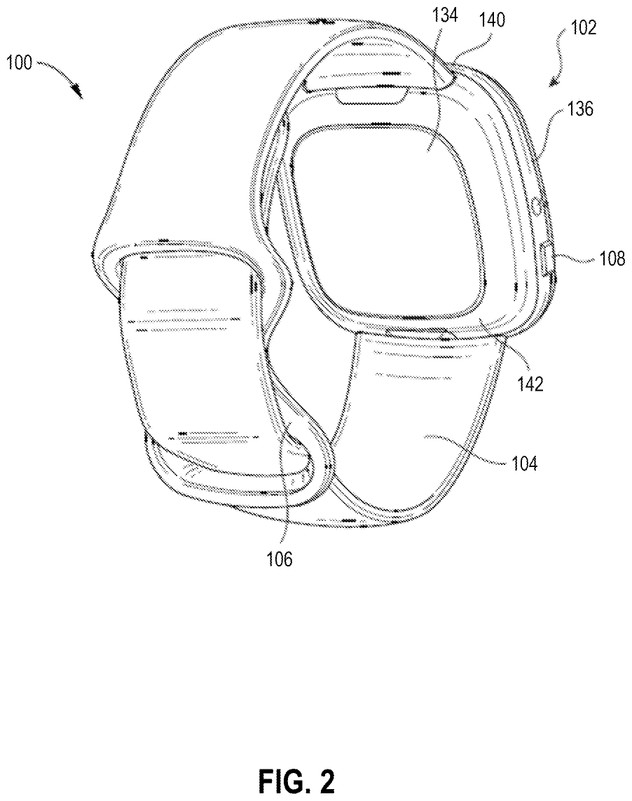

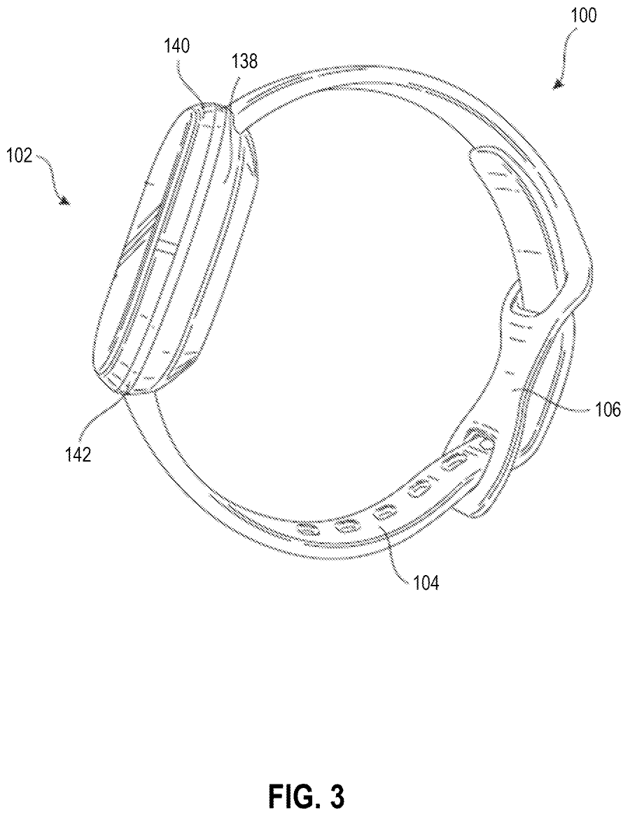

In one or more embodiments, the computing device described above and below according to example embodiments of the present disclosure can constitute, include, be coupled to, and/or otherwise be associated with one or more computing devices and/or computing systems described below and illustrated in the example embodiments depicted in FIGS. 1, 2, 3, 4, 5, and/or 6. For example, in at least one embodiment, the computing device described above and below according to example embodiments of the present disclosure can constitute, include, be coupled to, and/or otherwise be associated with wearable device 100, 100a, 100b, and/or 100c, external computing device 504, 504a, 504b, and/or 504c, and/or server system 604.

In the above embodiment, wearable device 100, 100a, 100b, and/or 100c, external computing device 504, 504a, 504b, and/or 504c, and/or server system 604 can individually and/or collectively perform the physiological monitoring and/or the health, wellness, and/or well-being assessment operations described herein (e.g., the physical, mental, emotional, behavioral, and/or sleep quality assessment operations) in accordance with one or more embodiments of the present disclosure. In this embodiment, based at least in part on (e.g., in response to) performing such assessment operations, wearable device 100, 100a, 100b, and/or 100c, external computing device 504, 504a, 504b, and/or 504c, and/or server system 604 can further perform, individually and/or collectively, one or more operations described herein that can facilitate alteration (e.g., improvement) of a user's health quality in accordance with one or more embodiments of the present disclosure.

In at least one embodiment of the present disclosure, to identify a correlation or an absence of correlation between a trigger event and at least one mood of a user at a defined time associated with the trigger event, the computing device can perform operations that can include, but are not limited to: detecting a trigger event associated with physiological data of a user; presenting one or more mood states to the user for selection based at least in part on detecting the trigger event, the one or more mood states corresponding to at least one mood experienced by the user at a defined time associated with the trigger event; annotating the physiological data with one or more annotations indicative of the at least one mood based at least in part on selection of the one or more mood states by the user; and training a model (e.g., a machine learning (ML) and/or artificial intelligence (AI) model) based at least in part on the one or more annotations such that the model identifies a correlation or an absence of correlation between the trigger event and the at least one mood. In this or another embodiment, the computing device can further implement the model to identify one or more other correlations or absences of correlation between one or more other trigger events and one or more other moods experienced by the user at each of one or more other defined times respectively associated with such other trigger event(s).

In one or more embodiments, the computing device can detect a trigger event associated with a user's physiological data that can be captured by one or more sensors (e.g., physiological sensors) of, for instance, a wearable physiological monitoring device (e.g., a wrist-worn device, a chest strap device) according to example embodiments described herein and/or another physiological monitoring device. The computing device can obtain such physiological data from such a wearable physiological monitoring device by using, for instance, a network (e.g., the Internet) as described in example embodiments of the present disclosure. In at least one embodiment, such physiological data can constitute, include, and/or otherwise be associated with, for instance: heart rate (HR) data, motion data (e.g., accelerometer data), respiration rate data, blood pressure data, blood oxygenation level data, body temperature data, data associated with (e.g., indicative or descriptive of) the user's deoxyribonucleic acid (DNA), electrodermal activity (EDA) data, stress related data, sleep data (e.g., sleep duration, time in sleep stages, metrics derived from profiling of user's heartrate during sleep events, etc.) and/or other physiological data that can be captured by, for instance, a wearable physiological monitoring device (e.g., a wrist-worn device, a chest strap device) according to example embodiments described herein and/or another physiological monitoring device.

In example embodiments described herein, the trigger event can constitute, include, and/or otherwise be associated with, for example: a defined physiological event (e.g., relatively depressed heart rate (HR) while awake, relatively elevated heart rate while at rest); a defined activity event (e.g., relatively less active or more active than usual); a defined sleep event (e.g., relatively better or worse sleep than usual); a defined behavioral event (e.g., relatively sedentary behavior when failing to satisfy a predefined sedentary step goal of a defined number of steps for a defined number of consecutive hours); a defined exercise event (e.g., workout routine); a defined mood logging event (e.g., predefined and/or regularly scheduled request for the user to input at least one mood the user is currently experiencing at the time of the request); and/or another event.

In one or more embodiments, as described above, the user can experience the at least one mood at a defined time associated with the trigger event. In one embodiment, the defined time can coincide with the trigger event (e.g., the defined time can occur at the same time the trigger event occurs) such that the user experiences the at least one mood at the same time the trigger event occurs. For example, in one embodiment, the trigger event can correspond to a relatively elevated at rest heart rate of the user (e.g., relative to historical at rest heart rate data of the user). In this embodiment, based at least in part on (e.g., in response to) detecting the user's relatively elevated at rest heart rate, the computing system can prompt the user to log (e.g., record, document) how they feel at the time the user is experiencing the relatively elevated at rest heart rate. For instance, in this embodiment, based at least in part on (e.g., in response to) detecting the user's relatively elevated at rest heart rate, the computing system can present the one or more mood states to the user for selection and the user can input the at least one mood the user is feeling at the time the user is experiencing the relatively elevated at rest heart rate.

In another embodiment, the defined time can be a certain time (e.g., 1 minute, 5 minutes, 15 minutes) after the trigger event occurs such that the user experiences the at least one mood after the trigger event occurs. For example, in at least one embodiment, a trigger event as referenced herein can constitute, include, and/or correspond to a defined activity that can be performed by the user. In this embodiment, the trigger event can constitute, include, and/or correspond to a defined activity such as, for instance: the above-described defined activity event (e.g., relatively less active or more active than usual); the above-described defined sleep event (e.g., relatively better or worse sleep than usual); the above-described defined exercise event (e.g., workout routine); and/or another defined activity that can be performed by the user.

In one embodiment, the defined activity can be a defined exercise event (e.g., workout routine). In this embodiment, based at least in part on (e.g., in response to) detecting one or more certain data in the user's physiological data that can be indicative of the user performing the defined exercise event (e.g., in response to detecting a relatively elevated respiratory rate, relatively elevated body temperature, relatively elevated blood oxygenation level), the computing system can prompt the user to log (e.g., record, document) how they feel at a certain time (e.g., 1 minute, 5 minutes, 15 minutes) after the user completes the defined exercise event. For instance, in this embodiment, the computing system can monitor the one or more certain data described above to determine when the user has completed the defined exercise event (e.g., by detecting when the user's respiratory rate, body temperature, and/or blood oxygenation level return to values and/or a range that indicate the user is at rest, not exercising). In this embodiment, based at least in part on (e.g., in response to) determining that the user has completed the defined exercise activity, the computing system can present the one or more mood states to the user for selection at a certain time (e.g., 1 minute, 5 minutes, 15 minutes) after making such a determination and the user can input the at least one mood the user is feeling at such a time (e.g., 1 minute, 5 minutes, 15 minutes) after completing the defined exercise event.

In at least one embodiment, to present the one or more mood states to the user for selection based at least in part on (e.g., in response to) detecting the trigger event, the computing device can generate, configure, and/or render an interactive user interface on a display (e.g., monitor, screen, touch screen, capacitive touch screen, resistive touch screen) that can be coupled to the computing device according to example embodiments of the present disclosure. In this and/or another embodiment, such an interactive user interface can include one or more interactive user interface elements (e.g., interactive buttons, data fields, drop-down menus) that can respectively correspond to the one or more mood states. In this and/or another embodiment, the computing device can generate, configure, and/or render such interactive user interface element(s) such that they can each receive input (e.g., a touch by the user, textual data) from the user that is indicative of a selection by the user of a mood state of the one or more mood states.

By way of example, in one embodiment, the computing device can generate, configure, and/or render an interactive user interface such as, for instance, an interactive button wheel on a touch screen coupled to the computing device. In this and/or another embodiment, the computing device can generate, configure, and/or render the interactive button wheel such that it has multiple interactive buttons (e.g., 5, 10, 15, 20) that are each labeled with a certain mood state of the one or more mood states. In this and/or another embodiment, each of such interactive buttons can be configured by the computing device such that they can receive input from the user by way of a touch (e.g., fingertip touch) by the user to indicate a selection by the user of the mood state labeled on the interactive button.

In some embodiments, the computing device can generate, configure, and/or render the above-described interactive user interface as a primary interactive user interface (e.g., a primary interactive button wheel) having one or more primary interactive user interface elements (e.g., primary interactive button(s)) that respectively correspond to one or more primary mood states (e.g., general mood state(s)). In these embodiments, based at least in part on (e.g., in response to) a selection by the user of one or more of such primary mood state(s) from the primary interactive user interface, the computing device can generate, configure, and/or render a secondary interactive user interface (e.g., a secondary interactive button wheel) that can constitute a sub-level of the primary interactive user interface. In these embodiments, the computing device can generate, configure, and/or render the secondary interactive user interface such that it has one or more secondary interactive user interface elements (e.g., secondary interactive button(s)) that respectively correspond to one or more secondary mood states. For example, in these embodiments, such secondary mood state(s) can constitute one or more mood sub-states that can be relatively more specific and/or granular compared to the relatively more general primary mood state selected by the user from the primary interactive user interface.

In example embodiments of the present disclosure, the one or more mood states (e.g., including the primary and/or secondary mood states described above) corresponding to the at least one mood experienced by the user at a defined time associated with the trigger event, as well as the at least one mood experienced by the user at the defined time associated with the trigger event, can constitute and/or include, but are not limited to, moods such as, for example: surprised, excited, energetic, hopeful, confident, happy, content, peaceful, tired, confused, sad, angry, tense, stressed, fearful, and/or another mood state. In some embodiments, the user may experience a mood that is not listed above. In these embodiments, the computing device can present the user with a “none apply” option for selection by the user. Additionally, and/or alternatively, in these embodiments, the computing device can also allow for the user to input a mood state that does apply but is not listed above (e.g., the user can input such a mood state into a user interface input element generated and/or rendered by the computing device on a display coupled to the computing device).

In some embodiments, the one or more mood states can respectively correspond to a certain level of arousal (e.g., level of energy) and/or a certain valence state that can be experienced by the user in connection with a certain trigger event and/or in connection with experiencing one or more of such mood states. In these embodiments, the level of arousal associated with a certain mood state selected by the user can provide insight (e.g., information, understanding) into the level of energy the user feels in connection with a certain trigger event and/or in connection with experiencing such a mood state. Similarly, in these embodiments, the valence state associated with a certain mood state selected by the user can provide insight (e.g., information, understanding) into the degree of attraction or aversion the user feels toward a certain trigger event and/or in connection with experiencing such a mood state.

In one embodiment, mood states including, for instance, surprised, excited, energetic, tense, stressed, and/or fearful can correspond to a relatively high level of arousal (e.g., relative to other levels of arousal felt by the user). In another embodiment, mood states including, for instance, hopeful, confident, happy, sad, and/or angry can correspond to a relatively moderate level of arousal (e.g., relative to other levels of arousal felt by the user).

In another embodiment, mood states including, for instance, content, peaceful, tired, and/or confused can correspond to a relatively low level of arousal (e.g., relative to other levels of arousal felt by the user).

In one embodiment, mood states including, for instance, excited, energetic, hopeful, confident, happy, content, and/or peaceful can correspond to a relatively positive valence state (e.g., relative to other valence states felt by the user). In another embodiment, mood states including, for instance, surprised can correspond to a relatively neutral valence state (e.g., relative to other valence states felt by the user). In another embodiment, mood states including, for instance, tired, confused, sad, angry, tense, stressed, and/or fearful can correspond to a relatively negative valence state (e.g., relative to other valence states felt by the user).

In example embodiments, as described above, the computing system can annotate the physiological data of the user with one or more annotations that can be indicative of the at least one mood experienced by the user at the defined time associated with the trigger event based at least in part on selection of the one or more mood states by the user. In at least one embodiment, the one or more annotations can each constitute, include, and/or otherwise be associated with one or more metadata, identifiers, and/or tags that can be indicative of, correspond to, and/or otherwise be associated with the at least one mood experienced by the user at the defined time associated with the trigger event.

In one embodiment, to annotate the physiological data of the user with the one or more annotations, the computing system can annotate one of one or more physiological data values (e.g., metadata values) of the physiological data with the one or more annotations (e.g., metadata, identifiers, tags) that can be indicative of the at least one mood experienced by the user at the defined time associated with the trigger event. Additionally, and/or alternatively, in this and/or another embodiment, the computing system can annotate a vector representation (e.g., metadata of a vector representation) of the physiological data with the one or more annotations (e.g., metadata, identifiers, tags) that can be indicative of the at least one mood experienced by the user at the defined time associated with the trigger event. Additionally, and/or alternatively, in this and/or another embodiment, the computing system can annotate a function (e.g., metadata of the function) that can be implemented (e.g., executed, run, calculated, computed) to generate such a vector representation of the physiological data. In this and/or another embodiment, the computing system can annotate such a function (e.g., metadata of the function) with the one or more annotations (e.g., metadata, identifiers, tags) that can be indicative of the at least one mood experienced by the user at the defined time associated with the trigger event. For example, the computing system interacts with a database (internal or external to the computing system) to store the physiological data along with the associated annotations.

In some embodiments, the level of arousal and/or valence state associated with each mood state selected by the user can provide one or more insights (e.g., information) that can be learned and/or used by the computing device (e.g., learned and/or used by a machine learning (ML) and/or artificial intelligence (AI) model implemented by the computing device) to perform one or more operations according to example embodiments described herein. In some embodiments, the computing device (e.g., via an ML and/or AI model) can learn and/or use such insight(s) and/or the above-described annotation(s) of the physiological data of the user to perform one or more operations according to example embodiments described herein. For example, in some embodiments, the computing device can use such insight(s) and/or the annotation(s) of the user's physiological data to train a machine learning (ML) and/or artificial intelligence (AI) model (e.g., a function, algorithm, process) that can then be implemented (e.g., executed, run) by the computing device to perform one or more operations according to example embodiments described herein. For instance, in these embodiments, the computing device can use such insight(s) and/or annotation(s) of the user's physiological data to train and/or implement an ML and/or AI model (e.g., a function, algorithm, process) that can include, but is not limited to, a classifier (e.g., nearest neighbor, random forest, support vector machine, decision tree, linear discriminant classifier), a neural network, a convolutional neural network, a hierarchical clustering algorithm, a pairwise and/or multidimensional pairwise model, and/or another ML and/or AI model.

In one embodiment, to train an ML and/or AI model to identify (e.g., infer, predict) the correlation or absence of correlation between the trigger event and the at least one mood experienced by the user at the defined time associated with the trigger event, the computing device can monitor (e.g., track) the user's physiological data over a defined period of time (e.g., 1 day, 1 week, 1 month, 3 months, 6 months, 1 year) to detect a plurality of trigger events associated with the user's physiological data. In this embodiment, based at least in part on (e.g., in response to) detecting the plurality of trigger events (e.g., including the trigger event described above) over such a defined period of time, the computing device can annotate the user's physiological data with a plurality of annotations (e.g., including the annotation(s) described above) that can each be indicative of one or more moods (e.g., including the at least one mood described above) experienced by the user at each of a plurality of defined times (e.g., including the defined time described above) that can be respectively associated with the plurality of trigger events.

In the above embodiment, based at least in part on (e.g., in response to) monitoring (e.g., tracking) the user's physiological data over such a defined period of time, the computing system can generate an annotated physiological dataset that can include such plurality of annotations (e.g., including the annotation(s) described above). In this embodiment, the computing device can use the annotated physiological dataset and/or the insight(s) described above to train an ML and/or AI model (e.g., one or more models provided above) to identify (e.g., infer, predict) the correlation or absence of correlation between the trigger event and the at least one mood experienced by the user at the defined time associated with the trigger event. In some embodiments, the computing device can use the annotated physiological dataset and/or the insight(s) described above to train an ML and/or AI model (e.g., one or more models provided above) to identify (e.g., infer, predict) any number of a plurality of correlations (e.g., including the correlation described above) and/or any number a plurality of absences of correlation (e.g., including the absence of correlation described above) between a plurality of moods experienced by the user at a plurality of defined times respectively associated with a plurality of trigger events associated with the user's physiological data.

In additional and/or alternative embodiments, as described above, a trigger event as referenced herein can constitute, include, and/or correspond to a defined activity (e.g., defined activity event, defined sleep event, defined exercise event) that can be performed by the user. For example, in these embodiments, the trigger event can constitute, include, and/or correspond to a defined activity such as, for instance, a defined sleeping event (e.g., relatively better or worse sleep than usual) or a defined exercise event (e.g., yoga, jogging, briskly walking, swimming) that can be performed by the user. In these embodiments, as described above, based at least in part on (e.g., in response to) determining that the user has completed the defined activity, the computing system can prompt the user to log (e.g., record, document) how they feel at a defined time (e.g., 1 minute, 5 minutes, 15 minutes) after the user has completed the defined activity. In these embodiments, based at least in part on (e.g., in response to) receiving input (e.g., via the interactive user interface described above) from the user that can be indicative of at least one mood experienced by the user at the defined time (e.g., 1 minute, 5 minutes, 15 minutes) after the user has completed the defined activity, the computing system can annotate the user's physiological data with one or more annotations that can be indicative of the at least one mood experienced by the user at such a defined time.

In the additional and/or alternative embodiments above, the computing device can monitor (e.g., track) the user's physiological data over a defined period of time (e.g., 1 day, 1 week, 1 month, 3 months, 6 months, 1 year) to detect a plurality of trigger events associated with the user's physiological data, including one or more defined activities that can be performed by the user. In these embodiments, based at least in part on (e.g., in response to) detecting the plurality of trigger events, which can include the one or more defined activities that can be performed by the user over such a defined period of time, the computing device can annotate the user's physiological data with a plurality of annotations that can each be indicative of one or more moods experienced by the user at each of one or more defined times that can be respectively associated with the one or more defined activities that can be performed by the user. In these embodiments, based at least in part on (e.g., in response to) monitoring (e.g., tracking) the user's physiological data over such a defined period of time, the computing system can generate the above-described annotated physiological dataset such that it can include such plurality of annotations associated with the one or more defined activities that can be performed by the user.

In the additional and/or alternative embodiments above, the computing device can use the annotated physiological dataset and/or the insight(s) described above to train an ML and/or AI model (e.g., one or more models provided above) to identify (e.g., infer, predict) a correlation or an absence of correlation between a defined activity that can be performed by the user (e.g., a defined activity of the one or more defined activities described above) and at least one mood (e.g., at least one mood of the one or more moods described above) experienced by the user at a defined time (e.g., a defined time of the one or more defined times described above) that can be associated with the defined activity. In some embodiments, the computing device can use the annotated physiological dataset and/or the insight(s) described above to train an ML and/or AI model (e.g., one or more models provided above) to identify (e.g., infer, predict) any number of a plurality of correlations (e.g., including the correlation described above) and/or any number a plurality of absences of correlation (e.g., including the absence of correlation described above) between a plurality of moods experienced by the user at a plurality of defined times respectively associated with a plurality of defined activities that can be performed by the user.

In example embodiments, based at least in part on (e.g., in response to) identifying (e.g., using an ML and/or AI model that can be trained as described above) a correlation or an absence of correlation between a trigger event and at least one mood experienced by the user at a defined time associated with the trigger event, the computing device can perform one or more operations according to one or more embodiments of the present disclosure. In these embodiments, as the trigger event can correspond to a defined activity that can be performed by the user, based at least in part on (e.g., in response to) identifying (e.g., using an ML and/or AI model) a correlation or an absence of correlation between the defined activity and at least one mood experienced by the user at a defined time (e.g., 1 minute, 5 minutes, 15 minutes) after the user has completed the defined exercise event associated with the defined activity, the computing device can perform one or more operations according to one or more embodiments of the present disclosure.

By way of example, in one or more embodiments, based at least in part on (e.g., in response to) identifying such a correlation or absence of correlation as described above, the computing device can perform operations that can include, but are not limited to, for instance: presenting the correlation or absence of correlation to the user and/or another computing device; providing the user and/or another computing device with an explanation of the correlation or absence of correlation such the user understands the connection, or lack thereof, between the trigger event, which can include a defined activity described above, and the at least one mood experienced by the user; suggesting one or more health improvement recommendations and/or engage another computing device to make such recommendation(s) based at least in part on (e.g., using) the correlation or absence of correlation (e.g., recommendation that the user perform or avoid performing a certain activity to experience or avoid experiencing the at least one mood, respectively); implementing one or more wellness promoting features and/or engage another computing device to implement such feature(s) based at least in part on (e.g., using) the correlation or absence of correlation (e.g., playing certain music and/or sounds to encourage the user to perform a certain activity to experience the at least one mood or to discourage the user from performing such an activity to avoid experiencing the at least one mood); and/or another operation according to one or more example embodiments of the present disclosure.

In one embodiment, based at least in part (e.g., in response to) identifying the correlation or absence of correlation, the computing device can, for example, generate an intelligent notification (e.g., a visual and/or audio notification) that can include and/or be indicative of the correlation or absence of correlation. In this or another embodiment, the computing device can further provide such an intelligent notification to the user and/or another computing device (e.g., a different computing device that is external to the computing device described above). For instance, in one embodiment, the computing device can provide the intelligent notification and/or the correlation or absence of correlation to the user using one or more data output devices such as, for example, a display device (e.g., a monitor, screen, display) and/or a speaker that can be included in, coupled to, and/or otherwise associated with the computing device.

In another embodiment described herein, the computing device can provide the above-described intelligent notification and/or the correlation or absence of correlation to another computing device (e.g., an external and/or remote computing device) such as, for instance, a client computing device, a computer, a laptop, a tablet, a smartphone, a physiological monitoring device, a wearable computing device, a wearable physiological monitoring device (e.g., a wrist-worn device, a chest strap device). In some embodiments, the computing device can provide the intelligent notification and/or the correlation or absence of correlation to another computing device and/or computing entity (e.g., module, model, algorithm, agent) that can function as and/or be associated with a medical and/or health counseling professional (e.g., a medical doctor, psychiatrist, mental health counselor).

In at least one embodiment of the present disclosure, based at least in part (e.g., in response to) identifying the correlation or absence of correlation, the computing device can, for example, generate one or more recommendations based at least in part on (e.g., using) the correlation or absence of correlation. For example, in this or another embodiment, the computing device can use the correlation or absence of correlation to generate a recommendation that the user perform a defined health improvement activity (e.g., the above-described defined activity, meditation, exercise, change of diet) to experience the at least one mood experienced by the user in connection with the trigger event or to avoid experiencing the at least one mood. In this or another embodiment, the computing device can further provide an intelligent notification (e.g., a visual and/or audio notification) that can include and/or be indicative of the correlation or absence of correlation and/or such one or more recommendations to the user and/or another computing device (e.g., a different computing device that is external to the computing device described above). For instance, in this or another embodiment, the computing device can provide, to the user and/or another computing device, an intelligent notification that can include and/or be indicative of the correlation or absence of correlation and/or the defined health improvement activity recommendation in the same manner as described above.

In at least one embodiment described herein, based at least in part (e.g., in response to) identifying the correlation or absence of correlation, the computing device can, for example, implement and/or facilitate implementation of one or more wellness promoting features of the computing device and/or another computing device (e.g., a different computing device that is external to the computing device described above) based at least in part on the correlation or absence of correlation. For instance, in this or another embodiment, the computing device can implement and/or facilitate implementation of one or more wellness promoting features of the computing device and/or another computing device at the defined time described above that can be associated with the trigger event (e.g., when the computing device detects the trigger event, at a certain time after the user completes a defined activity described above) and/or at a predefined time (e.g., each morning, each evening). For example, if the computing device had identified a correlation between a trigger event and at least one mood, it may implement the wellness promoting feature when the trigger event is again detected. Implementing the wellness promoting feature may comprise activating a feature of specific unit of the computing device and/or activating a feature of at least one external device as described below.

In one embodiment of the present disclosure, the computing device can implement (e.g., initiate, run, operate) one or more wellness promoting features that can be included with the computing device such as, for instance, a wellness promoting audio feature (e.g., by playing wellness promoting music and/or sounds), a wellness promoting lighting feature (e.g., by initiating a “sleep mode” and/or “night mode” of the computing device to dim one or more light sources of the computing device such as a screen, display, or monitor), and/or another wellness promoting feature of the computing device. For example, in this or another embodiment, the computing device can cause an audio system of the computing device to play wellness promoting music and/or sounds and/or cause a lighting system of the computing device to initiate a “sleep mode” and/or “night mode” to dim one or more light sources of the computing device such as a screen, display, or monitor.

In another embodiment of the present disclosure, the computing device can facilitate implementation of one or more wellness promoting features of another computing device such as, for instance: a wellness promoting exercise feature of a smart exercise system (e.g., an intelligent exercise machine included in, coupled to, and/or operated by another computing device); a wellness promoting audio feature of a smart audio system (e.g., a home audio system included in, coupled to, and/or operated by another computing device); a wellness promoting lighting feature of a smart lighting system (e.g., a home lighting system included in, coupled to, and/or operated by another computing device); a wellness promoting ambient temperature feature of a smart heating, ventilation, and air conditioning (HVAC) system (e.g., a home HVAC system coupled to and/or operated by another computing device); and/or another wellness promoting feature of another computing device. For instance, in this or another embodiment, the computing device can send instructions to one or more of the above-described smart systems that, when executed by such system(s) (e.g., via one or more processors), can cause the system(s) to perform operations to implement one or more wellness promoting features of such system(s).

In one embodiment of the present disclosure, the computing device can send instructions to the above-described smart exercise system that, when executed by such a system (e.g., via one or more processors), can cause it to operate in a certain mode or setting and/or to provide a recommendation to the user to select such a mode or setting. In another embodiment of the present disclosure, the computing device can send instructions to the above-described smart audio system that, when executed by such a system (e.g., via one or more processors), can cause it to play wellness promoting music and/or sounds. In another embodiment, the computing device can send instructions to the above-described smart lighting system that, when executed by such a system (e.g., via one or more processors), can cause it to initiate a “sleep mode” and/or “night mode” to dim one or more light sources (e.g., light bulbs) of the smart lighting system. In another embodiment, the computing device can send instructions to the above-described smart HVAC system that, when executed by such a system (e.g., via one or more processors), can cause it to output air at a certain wellness promoting temperature (e.g., a certain temperature that can be defined by the user).

In at least one embodiment described herein, the computing device can record, in a database (e.g., in a log that can be stored on a memory device), the above-described annotated physiological dataset that can include a plurality of correlations and a plurality of absences of correlation between a plurality of moods experienced by the user at a plurality of defined times respectively associated with a plurality of trigger events, which can include a plurality of defined activities that can be performed by the user. In some embodiments, the computing device can obtain and/or record, in such a database, one or more other correlations (e.g., a correlation value) or absence of correlations corresponding respectively to one or more other users. In these or other embodiments, the computing device can compare the correlation or absence of correlation of the user to the other correlation(s) or absence of correlation(s) corresponding respectively to the other user(s). In these or other embodiments, the computing device can further classify the user in one or more defined correlation categories (e.g., a category including correlations between a certain exercise and a certain mood) or one or more defined absence of correlation categories (e.g., a category including absences of correlation between a certain exercise and a certain mood) based at least in part on comparison of the correlation or absence of correlation of the user to the other correlation(s) or absence of correlation(s) corresponding respectively to the other user(s). In some embodiments, to perform the comparison and/or classification operations described above, the computing system can use one or more of the above-described ML and/or AI models (e.g., a classifier) that can perform comparison and/or classification operations described above.

In some embodiments, based at least in part on (e.g., in response to) classifying the user in one or more defined correlation categories or defined absence of correlation categories as described above, the computing device can perform one or more operations in accordance with one or more embodiments of the present disclosure. By way of example, in one or more embodiments, based at least in part on (e.g., in response to) classifying the user in a defined correlation category or a defined absence of correlation category as described above, the computing system can perform operations that can include, but are not limited to, for instance: informing (e.g., via an intelligent notification described above) the user and/or another computing device of such a classification; providing (e.g., via an intelligent notification described above) the user and/or another computing device with an explanation of such a classification of the user such the user understands why they are classified in such a category; suggesting one or more health improvement recommendations and/or engage another computing device to make such recommendation(s) based at least in part on (e.g., using) such a classification of the user (e.g., recommendation that the user perform or avoid performing a certain activity to experience or avoid experiencing the at least one mood, respectively); implementing one or more wellness promoting features and/or engage another computing device to implement such feature(s) based at least in part on (e.g., using) such a classification of the user (e.g., playing certain music and/or sounds to encourage the user to perform a certain activity to experience the at least one mood or to discourage the user from performing such an activity to avoid experiencing the at least one mood); and/or another operation according to one or more example embodiments of the present disclosure.

In some embodiments, based at least in part on (e.g., in response to) identifying the correlation or absence of correlation and/or classifying the user in one or more defined correlation categories or defined absence of correlation categories as described above, the computing system can create one or more health improvement plans and/or systems (e.g., holistic and/or lifestyle plan(s) and/or system(s)) that the computing device can recommend to the user for implementation. In these embodiments, the computing system can create such health improvement plan(s) and/or system(s) such that they are specific to the user (e.g., customized for the user's holistic and/or lifestyle goals). In these embodiments, in creating and/or facilitating implementation of such health improvement plan(s) and/or system(s) on behalf of the user, the computing device can perform any of the operations described above and/or in one or more embodiments of the present disclosure to assist the user in improving their emotional and/or mental well-being and/or achieving a healthy and/or holistic lifestyle.

Example aspects of the present disclosure provide several technical effects, benefits, and/or improvements in computing technology. For instance, a computing device according to example embodiments of the present disclosure can identify a correlation or an absence of correlation between a trigger event associated with physiological data of a user and at least one mood of the user at a defined time associated with the trigger event. In these embodiments, the computing device can use such a correlation or absence of correlation to perform one or more operations that can facilitate alteration (e.g., improvement) of the user's health quality. Furthermore, in some embodiments, the computing device can perform one or more actions based on the trained model to help improve the user's mood. For instance, if computing device by performing one or more actions. For instance, the actions can include prompting the user to perform a mindfulness session (e.g., guided breathing) when the user is determined to be angry. Alternatively, the actions can include prompting the user to exercise if the user's mood is sad.

In some embodiments, by identifying and using such a correlation or absence of correlation described above, the computing device can accurately and consistently determine which trigger events associated with the physiological data of the user cause the user to experience which moods. In these embodiments, by accurately and consistently determine which trigger events associated with the physiological data of the user cause the user to experience which moods, the computing device can thereby reduce the processing workload of one or more processors that execute operations to make such a determination. For example, in these or other embodiments, the computing device can thereby reduce the processing workload of one or more processors that can be included in and/or coupled to the computing device and/or another computing device that is external to the computing device such as, for instance, another computing device and/or computing entity (e.g., module, model, algorithm, agent) that can function as and/or be associated with a medical and/or health counseling professional (e.g., a processor of another computing device that can be used to conduct mental and/or emotional health studies, diagnosis various mental and/or emotional health conditions, suggest mental and/or emotional health improvement activities, plans, and/or systems). In these or other embodiments, by reducing the processing workload of such one or more processors, the computing device can thereby improve the processing efficiency and/or processing performance of the processor(s), as well as reduce computational costs of the processor(s).

### Example Devices and Systems

FIGS. 1, 2, and 3 each illustrate a perspective view of an example, non-limiting wearable device 100 according to one or more example embodiments of the present disclosure. In example embodiments described herein, wearable device 100 can constitute and/or include a wearable computing device. For instance, in these or other example embodiments, wearable device 100 can constitute and/or include a wearable computing device such as, for example, a wearable physiological monitoring device that can be worn by a user (also referred to herein as a “wearer”) and/or capture one or more types of physiological data of the user (e.g., heart rate (HR) data, motion data (e.g., accelerometer data), body temperature data, respiration rate data, blood pressure data, blood oxygenation level data, deoxyribonucleic acid (DNA) data, electrodermal activity (EDA) data, stress related data).

Wearable device 100 according to example embodiments of the present disclosure can include a display 102, an attachment component 104, a securement component 106, and a button 108 that can be located on a side of wearable device 100. In at least one embodiment, two sides of display 102 can be coupled (e.g., mechanically, operatively) to attachment component 104. In some embodiments, securement component 106 can be located on, coupled to (e.g., mechanically, operatively), and/or integrated with attachment component 104. In these or other embodiments, securement component 106 can be positioned opposite display 102 on an opposing end of attachment component 104. In some embodiments, button 108 can be located on a side of wearable device 100, underneath display 102.

Display 102 according to example embodiments described herein can constitute and/or include any type of electronic display or screen known in the art. For example, in some embodiments, display 102 can constitute and/or include a liquid crystal display (LCD) or organic light emitting diode (OLED) display such as, for instance, a transmissive LCD display or a transmissive OLED display. Display 102 according to example embodiments can be configured to provide brightness, contrast, and/or color saturation features according to display settings that can be maintained by control circuitry and/or other internal components and/or circuitry of wearable device 100. In some embodiments, display 102 can constitute and/or include a touchscreen such as, for instance, a capacitive touchscreen. For example, in these embodiments, display 102 can constitute and/or include a surface capacitive touchscreen or a projective capacitive touch screen that can be configured to respond to contact with electrical charge-holding members or tools, such as a human finger.

In some embodiments, display 102 can be configured to provide (e.g., render) a variety of information such as, for example, the time, the date, body signals (e.g., physiological data of a user wearing wearable device 100), readings based upon user input, and/or other information. In one embodiment, such body signals can include, but are not limited to, heart rate data (e.g., heart beats per minute), motion data (e.g., movement data, accelerometer data), blood pressure data, body temperature data, respiration rate data, blood oxygenation level data, deoxyribonucleic acid (DNA) data, electrodermal activity (EDA) data, stress related data and/or any other body signal that one of ordinary skill in the art would understand that can be measured by a wearable device such as, for instance, wearable device 100. In some embodiments, the readings based upon user input can include, but are not limited to, the number of steps a user has taken, the distance traveled by the user, the sleep schedule of the user, travel routes of the user, elevation climbed by the user, and/or any other metric that one of ordinary skill in the art would understand that can be input by a user into a wearable device such as, for instance, wearable device 100.

In at least one embodiment of the present disclosure, the above-described body signals and/or readings based upon user input can be used to calculate further analytics to provide a user with data such as, for instance, a fitness score, a sleep quality score, a number of calories burned by the user, and/or other data. In some embodiments, wearable device 100 can take in (e.g., capture, collect, receive, measure) outside data irrespective of the user such as, for example: an ambient temperature of an environment surrounding and/or external to wearable device 100; an amount of sun exposure wearable device 100 is subjected to; an atmospheric pressure of the environment surrounding and/or external to wearable device 100; an air quality of the environment surrounding and/or external to wearable device 100; the location of wearable device 100 based on, for instance, a global positioning system (GPS); and/or other outside factors that one of ordinary skill in the art would understand a wearable device such as, for instance, wearable device 100 can take in (e.g., capture, collect, receive, measure).

Attachment component 104 according to example embodiments described herein can be used to attach (e.g., affix, fasten) wearable device 100 to a user of wearable device 100. In some embodiments, attachment component 104 can take the form of, for example, a strap, an elastic band, a rope, and/or any other form of attachment one of ordinary skill in the art would understand can be used to attach a wearable device such as, for instance, wearable device 100 to a user.

Securement component 106 according to example embodiments of the present disclosure can facilitate attachment of attachment component 104 upon a user of wearable device 100. In some embodiments, securement component 106 can include, but is not limited to, a pin and hole locking mechanism (e.g., a buckle), a magnet system, a lock, a clip, and/or any other type of securement that one of ordinary skill would understand can be used to facilitate attachment of a wearable device such as, for instance, wearable device 100 to a user. In one embodiment, wearable device 100 does not include securement component 106. For example, in this or another embodiment, wearable device 100 can be secured to a user with a strap that can be tied around the user's wrist and/or another suitable appendage.

Button 108 according to example embodiments described herein can allow for a user to interact with wearable device 100 and/or allow for the user to provide a form of input into wearable device 100. In the example embodiment depicted in FIGS. 1, 2, and 3, one button 108 is shown on wearable device 100. However, it should be appreciated that wearable device 100 is not so limiting. For example, in some embodiments, wearable device 100 can include any number of buttons that allow a user to further interact with wearable device 100 and/or to provide alternative inputs. In at least one embodiment, wearable device 100 does not include button 108. For instance, as described above, in example embodiments, wearable device 100 can include a screen such as, for example, a touch screen that can receive inputs through (e.g., by way of) the touch of the user. In additional or alternative embodiments, wearable device 100 can include a microphone that can receive inputs through (e.g., by way of) voice commands of a user.

In some embodiments, wearable device 100 can constitute a portable computing device that can be designed so that it can be inserted into a wearable case (e.g., as illustrated in the example embodiments depicted in FIGS. 1, 2, and 3). In some embodiments, wearable device 100 can constitute a portable computing device that can be designed so that it can be inserted into one or more of multiple different wearable cases (e.g., a wristband case, a belt-clip case, a pendant case, a case configured to be attached to a piece of exercise equipment such as a bicycle). Wearable device 100 according to embodiments described herein can be formed into one or more shapes and/or sizes to allow for coupling to (e.g., secured to, worn, borne by) the body or clothing of a user. In some embodiments, wearable device 100 can constitute a portable computing device that can be designed to be worn in limited manners such as, for instance, a computing device that is integrated into a wristband in a non-removable manner and/or can be intended to be worn specifically on a person's wrist (or perhaps ankle).

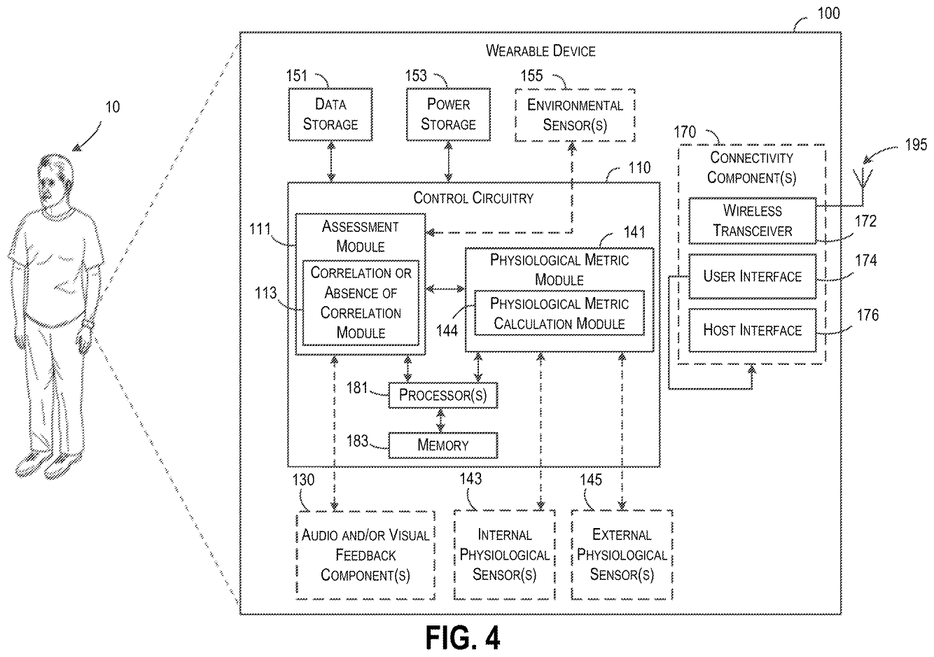

Irrespective of configuration, wearable device 100 according to example embodiments of the present disclosure can include one or more physiological and/or environmental sensors (e.g., internal physiological sensor(s) 143, external physiological sensor(s) 145, and/or environmental sensor(s) 155 described below with reference to FIG. 4) that can be configured to collect physiological and/or environmental data in accordance with various embodiments disclosed herein. In some embodiments, wearable device 100 can be configured to analyze and/or interpret collected physiological and/or environmental data to perform one or more health, wellness, and/or well-being assessments (e.g., physical, mental, emotional, behavioral, and/or sleep quality assessment(s)) of a user (e.g., a wearer) of wearable device 100 according to one or more embodiments described herein. In additional and/or alternative embodiments, wearable device 100 can be configured to communicate with another computing device or server that can perform such one or more health, wellness, and/or well-being assessments (e.g., physical, mental, emotional, behavioral, and/or sleep quality assessment(s)) of a user (e.g., a wearer) of wearable device 100 according to one or more embodiments described herein.

Wearable device 100 in accordance with one or more example embodiments of the present disclosure can include one or more physiological and/or environmental components and/or modules that can be designed to determine one or more physiological and/or environmental metrics associated with a user (e.g., a wearer) of wearable device 100. In at least one embodiment, such physiological and/or environmental component(s) and/or module(s) can constitute and/or include one or more physiological and/or environmental sensors. For instance, although not depicted in the example embodiments illustrated in FIGS. 1, 2, and 3, in some embodiments, wearable device 100 can include one or more physiological and/or environmental sensors such as, for example, an accelerometer, a heart rate sensor (e.g., photoplethysmography (PPG) sensor), an electrodermal activity (EDA) sensor, a body temperature sensor, an environment temperature sensor, and/or another physiological and/or environmental sensor. In these or other embodiments, such physiological and/or environmental sensor(s) can be disposed on, coupled to, and/or otherwise be associated with an underside and/or a backside (e.g., back 134) of wearable device 100.

In some embodiments, the above-described physiological and/or environmental sensor(s) can be disposed on, coupled to, and/or otherwise be associated with wearable device 100 such that the sensor(s) can be in contact with or substantially in contact with human skin when wearable device 100 is worn by a user. For example, in embodiments where wearable device 100 can be worn on a user's wrist, the physiological and/or environmental sensor(s) can be disposed on, coupled to, and/or otherwise be associated with back 134 that can be substantially opposite display 102 and touching an arm of the user. In one embodiment, the above-described physiological and/or environmental sensor(s) can be disposed on, coupled to, and/or otherwise be associated with an interior or skin-side of wearable device 100 (e.g., a side of wearable device 100 that contacts, touches, and/or faces the skin of the user such as, for instance, back 134 and/or bottom 142). In another embodiment, the physiological and/or environmental sensors can be disposed on one or more sides of wearable device 100, including the skin-side (e.g., back 134, bottom 142) and one or more sides (e.g., first side 136, second side 138, top 140, display 102) of wearable device 100 that face and/or are exposed to the ambient environment (e.g., the external environment surrounding wearable device 100).

FIG. 4 illustrates a block diagram of the above-described example, non-limiting wearable device 100 according to one or more example embodiments of the present disclosure. That is, for instance, FIG. 4 illustrates a block diagram of one or more internal and/or external components of the above-described example, non-limiting wearable device 100 according to one or more example embodiments of the present disclosure.

As described above with reference to the example embodiments depicted in FIGS. 1, 2, and 3, wearable device 100 can constitute and/or include a wearable computing device such as, for instance, a wearable physiological monitoring device. For example, in the example embodiment depicted in FIG. 4, wearable device 100 can constitute and/or include a wearable physiological monitoring device that can be worn by a user 10 (also referred to herein as a “wearer” or “wearer 10”) and/or can be configured to gather data regarding activities performed by user 10 and/or data regarding user's 10 physiological (e.g., physical), mental, and/or emotional state (e.g., including sleep quality). In this or another embodiment, such data can include data representative of the ambient environment around user 10 or user's 10 interaction with the environment. For example, in some embodiments, the data can constitute and/or include motion data regarding user's 10 movements, ambient light, ambient noise, air quality, and/or physiological data obtained by measuring various physiological characteristics of user 10 (e.g., heart rate, respiratory data, body temperature, blood oxygen levels, perspiration levels, movement data).

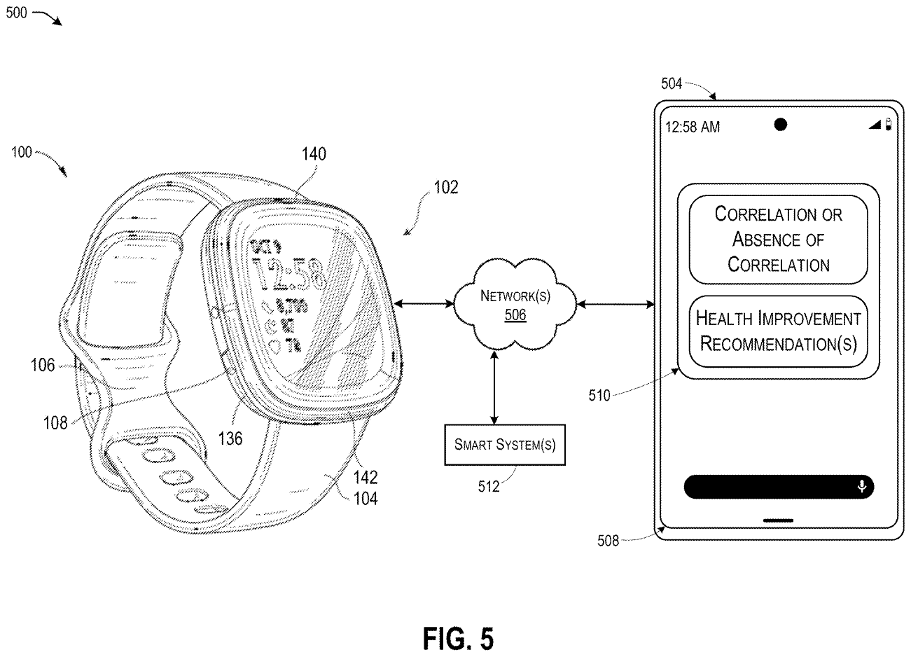

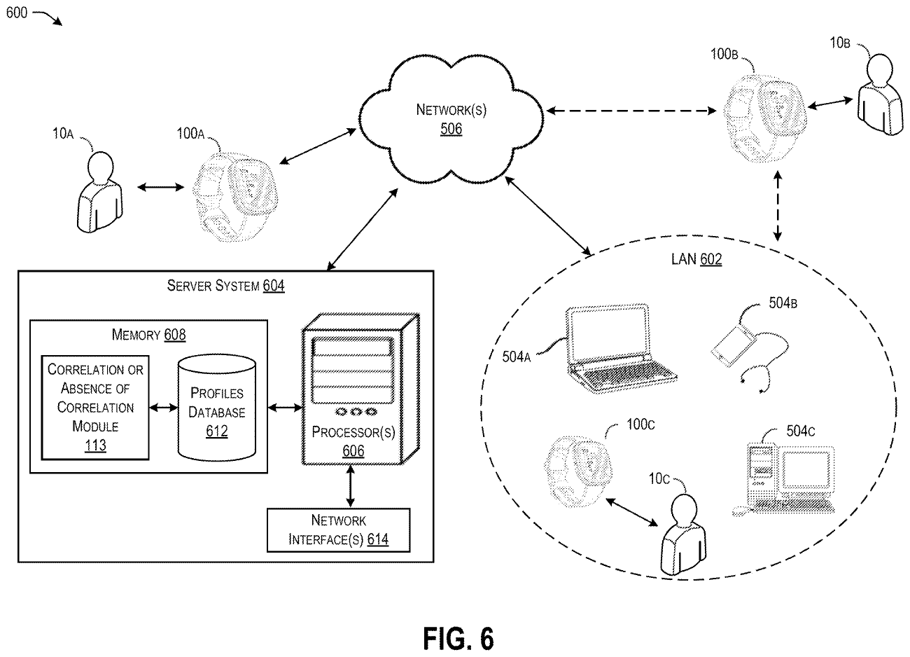

Although certain embodiments are disclosed herein in the context of wearable physiological monitoring devices, it should be appreciated that the present disclosure is not so limiting. For example, it should be understood that the physiological monitoring and the health, wellness, and/or well-being assessment principles and features disclosed herein (e.g., physical, mental, emotional, behavioral, and/or sleep quality assessment) can be applicable with respect to and/or implemented using any suitable or desirable type of computing device or combination of computing devices, whether wearable or not. For instance, the physiological monitoring and the health, wellness, and/or well-being assessment principles and features disclosed herein (e.g., physical, mental, emotional, behavioral, and/or sleep quality assessment) in accordance with one or more embodiments can by performed and/or implemented using any suitable or desirable type of computing device or combination of computing devices such as, for example, a client computing device, a laptop, a tablet, a server (e.g., server system 604 described below and depicted in FIG. 6), a wearable computing device (e.g., wearable device 100), a smartphone (e.g., external computing device 504 described below and depicted in FIG. 5), and/or another computing device, whether wearable or not.

As illustrated in FIG. 4, wearable device 100 according to example embodiments of the present disclosure can include one or more audio and/or visual feedback components 130 such as, for instance, electronic touchscreen display units, light-emitting diode (LED) display units, audio speakers, light-emitting diode (LED) lights, buzzers, and/or another type of audio and/or visual feedback module. In certain embodiments, one or more audio and/or visual feedback modules 130 can be located on and/or otherwise associated with a front side of wearable device 100 and/or display 102. For example, in wearable embodiments of wearable device 100, an electronic display such as, for instance, display 102 can be configured to be externally presented to user 10 viewing wearable device 100.

Wearable device 100 according to example embodiments of the present disclosure can include control circuitry 110. Although certain modules and/or components are illustrated as part of control circuitry 110 in the diagram of FIG. 4, it should be understood that control circuitry 110 associated with wearable device 100 and/or other components or devices in accordance with example embodiments of the present disclosure can include additional components and/or circuitry such as, for instance, one or more additional components of the illustrated components depicted in FIG. 4. Furthermore, in certain embodiments, one or more of the illustrated components of control circuitry 110 can be omitted and/or different than that shown in FIG. 4 and described in association therewith.

The term “control circuitry” is used herein according to its broad and/ordinary meaning and can include any combination of software and/or hardware elements, devices, and/or features that can be implemented in connection with operation of wearable device 100. Furthermore, the term “control circuitry” can be used substantially interchangeably in certain contexts herein with one or more of the terms “controller,” “integrated circuit,” “IC,” “application-specific integrated circuit,” “ASIC,” “controller chip,” or the like.

Control circuitry 110 according to example embodiments of the present disclosure can constitute and/or include one or more processors, data storage devices, and/or electrical connections. In one embodiment, control circuitry 110 can be implemented on a system on a chip (SoC), however, those skilled in the art will recognize that other hardware and/or firmware implementations are possible.

In one or more embodiments of the present disclosure, control circuitry 110 can constitute and/or include one or more processors 181 that can be configured to execute computer-readable instructions that, when executed, cause wearable device 100 to perform one or more operations. In at least one embodiment, control circuitry 110 can constitute and/or include processor(s) 181 that can be configured to execute operational code (e.g., instructions, processing threads, software) for wearable device 100 such as, for instance, firmware or the like. Processor(s) 181 according to example embodiments described herein can each be a processing device. For instance, in the example embodiment depicted in FIG. 4, processor(s) 181 can each be a central processing unit (CPU), microprocessor, microcontroller, integrated circuit (e.g., an application-specific integrated circuit (ASIC)), and/or another type of processing device. In this or another example embodiment, processor(s) 181 can be coupled to (e.g., electrically, communicatively, physically, operatively) to one or more components of control circuitry 110 and/or wearable device 100 such that processor(s) 181 can facilitate one or more operations in accordance with one or more example embodiments described herein.

In at least one embodiment of the present disclosure, the above-described computer-readable instructions and/or operational code that can be executed by processor(s) 181 can be stored in one or more data storage devices of wearable device 100. In the example embodiment depicted in FIG. 4, such computer-readable instructions and/or operational code can be stored in memory 183 of wearable device 100. In this or another example embodiment, memory 183 can be coupled to (e.g., electrically, communicatively, physically, operatively) to one or more components of control circuitry 110 and/or wearable device 100 such that memory 183 can facilitate one or more operations in accordance with one or more example embodiments described herein.

Memory 183 according to example embodiments described herein can store computer-readable and/or computer executable entities (e.g., data, information, applications, models, algorithms) that can be created, modified, accessed, read, retrieved, and/or executed by each of processor(s) 181. In some embodiments, memory 183 can constitute, include, be coupled to (e.g., operatively), and/or otherwise be associated with a computing system and/or media such as, for example, one or more computer-readable media, volatile memory, non-volatile memory, random-access memory (RAM), read only memory (ROM), hard drives, flash drives, and/or other memory devices. In these or other embodiments, such one or more computer-readable media can include, constitute, be coupled to (e.g., operatively), and/or otherwise be associated with one or more non-transitory computer-readable media. Although not depicted in the example embodiment illustrated in FIG. 4, in some embodiments, memory 183 can include (e.g., store) an assessment module 111, a correlation or absence of correlation module 113, physiological metric module 141, physiological metric calculation module 144, and/or other modules and/or data that can be used to facilitate one or more operations described herein.

Control circuitry 110 according to example embodiments of the present disclosure can constitute and/or include assessment module 111. Assessment module 111 according to example embodiments of the present disclosure can constitute and/or include one or more hardware and/or software components and/or features that can be configured to perform one or more assessments of user 10 in accordance with one or more embodiments described herein. For example, in some embodiments, assessment module 111 can constitute and/or include one or more hardware and/or software components and/or features that can be configured to perform one or more health, wellness, and/or well-being assessments (e.g., physical, mental, emotional, behavioral, and/or sleep quality assessment(s)) of user 10 according to one or more embodiments described herein. In some embodiments, to perform such assessment(s), assessment module 111 can use inputs from one or more environmental sensors 155 (e.g., ambient light sensor) and/or information from physiological metric module 141.

In certain embodiments, assessment module 111 can include a correlation or absence of correlation module 113 that can be configured to identify a correlation or absence of correlation between a trigger event associated with physiological data of user 10 and at least one mood experienced by user 10 at a defined time associated with the trigger event. In these and/or other embodiments, wearable device 100 can implement correlation or absence of correlation module 113 to identify such a correlation or absence of correlation between the trigger event and the at least one mood experienced by user 10 as described herein in accordance with one or more embodiments of the present disclosure.

In one embodiment, correlation or absence of correlation module 113 can constitute and/or include one or more of the ML and/or AI models described herein (e.g., a classifier) that can identify such a correlation or absence of correlation between the trigger event and the at least one mood experienced by user 10. In one embodiment, wearable device 100 can train such ML and/or AI model(s) as described herein using the above-described annotated physiological dataset. In one embodiment, wearable device 100 can implement (e.g., execute, run) such ML and/or AI model(s) to identify such a correlation or absence of correlation between the trigger event and the at least one mood experienced by user 10 using physiological data of user 10 that can be accumulated by assessment module 111 such as, for instance, the values of one or more physiological metrics (e.g., user's 10 heart rate, motion, temperature, respiration, perspiration, electrodermal activity (EDA)) that can be determined by physiological metric calculation module 144 of physiological metric module 141.

In some embodiments, based at least in part on (e.g., in response to) identifying such a correlation or absence of correlation between the trigger event and at least one mood experienced by user 10 at a defined time associated with the trigger event, wearable device 100 can perform one or more operations described herein to facilitate alteration (e.g., improvement) of user's 10 health, wellness, and/or well-being (e.g., physical, mental, emotional, behavioral, and/or sleep quality). For example, in at least one embodiment, wearable device 100 can perform operation(s) that can include, but not limited to: presenting the correlation or absence of correlation to user 10 and/or another computing device; providing user 10 and/or another computing device with an explanation of the correlation or absence of correlation such user 10 understands the connection, or lack thereof, between the trigger event, which can include a defined activity as described herein, and the at least one mood experienced by user 10; suggesting one or more health improvement recommendations and/or engage another computing device to make such recommendation(s) based at least in part on (e.g., using) the correlation or absence of correlation (e.g., recommendation that user 10 perform or avoid performing a certain activity to experience or avoid experiencing the at least one mood, respectively); implementing one or more wellness promoting features and/or engage another computing device to implement such feature(s) based at least in part on (e.g., using) the correlation or absence of correlation (e.g., playing certain music and/or sounds to encourage user 10 to perform a certain activity to experience the at least one mood or to discourage user 10 from performing such an activity to avoid experiencing the at least one mood); and/or another operation according to one or more example embodiments of the present disclosure.

In certain embodiments, physiological metric module 141 and/or physiological metric calculation module 144 can be communicatively coupled with one or more internal physiological sensors 143 that can be embedded and/or integrated in wearable device 100. In certain embodiments, physiological metric module 141 and/or physiological metric calculation module 144 can be optionally in communication with one or more external physiological sensors 145 not embedded and/or integrated in wearable device 100 (e.g., an electrode or sensor integrated in another electronic device). In some embodiments, examples of internal physiological sensors 143 and/or external physiological sensors 145 can constitute and/or include, but are not limited to, one or more sensors that can measure (e.g., capture, collect, receive) physiological data of user 10 such as, for instance, body temperature, heart rate, blood oxygen level, movement, respiration, perspiration, electrodermal activity (EDA), stress data, and/or other physiological data of user 10.

In the example embodiment depicted in FIG. 4, wearable device 100 can include one or more data storage components 151 (denoted as “data storage 151” in FIG. 4). Data storage component(s) 151 according to example embodiments can constitute and/or include any suitable or desirable type of data storage such as, for instance, solid-state memory, which can be volatile or non-volatile. In some embodiments, such solid-state memory of wearable device 100 can constitute and/or include any of a wide variety of technologies such as, for instance, flash integrated circuits, phase change (PC) memory, phase change (PC) random-access memory (RAM), programmable metallization cell RAM (PMC-RAM or PMCm), ovonic unified memory (OUM), resistance RAM (RRAM), NAND memory, NOR memory, EEPROM, ferroelectric memory (FeRAM), MRAM, or other discrete NVM (non-volatile solid-state memory) chips. In some embodiments, data storage component(s) 151 can be used to store system data, such as operating system data and/or system configurations or parameters. In some embodiments, wearable device 100 can include data storage utilized as a buffer and/or cache memory for operational use by control circuitry 110.

Data storage component(s) 151 according to example embodiments can include various sub-modules that can be implemented to facilitate the physiological monitoring and the health, wellness, and/or well-being assessment principles and features disclosed herein (e.g., physical, mental, emotional, behavioral, and/or sleep quality assessment) in accordance with one or more embodiments. For example, in at least one embodiment, data storage 151 can include one or more sub-modules that can include, but not limited to: an information collection module (e.g., physiological metric module 141, physiological metric calculation module 144) that can manage the collection of physiological and/or environmental data relevant to any health, wellness, and/or well-being assessment described herein (e.g., physical, mental, emotional, behavioral, and/or sleep quality assessment(s)); a heart rate determination module that can determine values and/or patterns of one or more types of heart rates of user 10; a trigger event detection module (e.g., assessment module 111, correlation or absence of correlation module 113, one or more ML and/or AI models described herein) that can detect a trigger event as described herein that can be associated with physiological data of user 10; a sleep detection module that can detect an attempt or onset of sleep by the user 10; a presentation module that can manage presentation of information to user 10 that can be associated with any health, wellness, and/or well-being assessment described herein (e.g., physical, mental, emotional, behavioral, and/or sleep quality assessment(s)); a feedback management module for collecting and interpreting any input data and/or feedback received from user 10 (e.g., information associated with user's 10 physical, mental, emotional, behavioral, and/or sleep quality state); and/or another sub-module.

Wearable device 100 according to example embodiments can further include a power storage module 153 (denoted as “power storage 153”), which can constitute and/or include a rechargeable battery, one or more capacitors, or other charge-holding device(s). In some embodiments, the power stored by power storage module 153 can be utilized by control circuitry 110 for operation of wearable device 100, such as for powering display 102. In some embodiments, power storage module 153 can receive power over a host interface of wearable device 100 (e.g., via one or more host interface circuitry and/or components 176 (denoted as “host interface 176” in FIG. 4)) and/or through other means.

Wearable device 100 according to example embodiments can further include one or more environmental sensors 155. In at least one embodiment, examples of such environmental sensors 155 can include, but are not limited to, sensors that can determine and/or measure, for instance, ambient light, external (non-body) temperature, altitude, device location (e.g., global-positioning system (GPS)), and/or another environmental data.

Wearable device 100 according to example embodiments can further include one or more connectivity components 170, which can include, for example, a wireless transceiver 172. Wireless transceiver 172 according to example embodiments can be communicatively coupled to one or more antenna devices 195, which can be configured to wirelessly transmit and/or receive data and/or power signals to and/or from wearable device 100 using, but not limited to, peer-to-peer, WLAN, and/or cellular communications. For example, wireless transceiver 172 can be utilized to communicate data and/or power between wearable device 100 and an external computing device (not illustrated in FIG. 4) such as, for instance, an external client computing device (e.g., a smartphone, tablet, computer) and/or an external host system (e.g., a server), which can be configured to interface with wearable device 100. In certain embodiments, wearable device 100 can include one or more host interface circuitry and/or components 176 (denoted as “host interface 176” in FIG. 4) such as, for instance, wired interface components that can communicatively couple wearable device 100 with the above-described external computing device (e.g., a smartphone, table, computer, server) to receive data and/or power therefrom and/or transmit data thereto.

Connectivity component(s) 170 according to example embodiments can further include one or more user interface components 174 (denoted as “user interface 174” in FIG. 4) that can be used by wearable device 100 to receive input data from user 10 and/or provide output data to user 10. In some embodiments, user interface component(s) 174 can be coupled to (e.g., operatively, communicatively) and/or otherwise be associated with audio and/or visual feedback component(s) 130. For instance, in these embodiments, display 102 of wearable device 100 can constitute and/or include a touchscreen display that can be configured to provide (e.g., render) output data to user 10 and/or to use audio and/or visual feedback component(s) 130 to receive user input through user contact with the touchscreen display. In some embodiments, user interface component(s) 174 can further constitute and/or include one or more buttons or other input components or features.

Connectivity component(s) 170 according to example embodiments can further include host interface circuitry and/or component(s) 176, which can be, for example, an interface that can be used by wearable device 100 to communicate with the above-described external computing device (e.g., a smartphone, table, computer, server) over a wired or wireless connection. Host interface circuitry and/or component(s) 176 according to example embodiments can utilize and/or otherwise be associated with any suitable or desirable communication protocol and/or physical connector such as, for instance, universal serial bus (USB), micro-USB, Wi-Fi, Bluetooth, FireWire, PCIe, or the like. For wireless connections, host interface circuitry and/or component(s) 176 according to example embodiments can be incorporated with wireless transceiver 172.

Although certain functional modules and components are illustrated and described herein, it should be understood that authentication management functionality in accordance with the present disclosure can be implemented using a number of different approaches. For example, in some embodiments, control circuitry 110 can constitute and/or include one or more processors (e.g., processor(s) 181) that can be controlled by computer-executable instructions that can be stored in a memory (e.g., memory 183, data storage component(s) 151) so as to provide functionality such as is described herein. In other embodiments, such functionality can be provided in the form of one or more specially designed electrical circuits. In some embodiments, such functionality can be provided by one or more processors (e.g., processor(s) 181) that can be controlled by computer-executable instructions that can be stored in a memory (e.g., memory 183, data storage component(s) 151) that can be coupled to (e.g., communicatively, operatively, electrically) one or more specially designed electrical circuits. Various examples of hardware that can be used to implement the concepts outlined herein can include, but are not limited to, application specific integrated circuits (ASICs), field-programmable gate arrays (FPGAs), and general-purpose microprocessors that can be coupled with memory that stores executable instructions for controlling the general-purpose microprocessors.

FIG. 5 illustrates a diagram of an example, non-limiting user assessment management system 500 according to one or more example embodiments of the present disclosure. User assessment management system 500 depicted in FIG. 5 illustrates an example, non-limiting networked relationship between wearable device 100, an external computing device 504, and/or one or more smart systems 512 in accordance with one or more embodiments.

With reference to the example embodiment described above and depicted in FIG. 4, wearable device 100 according to example embodiments of the present disclosure can perform one or more health, wellness, and/or well-being assessments (e.g., physical, mental, emotional, behavioral, and/or sleep quality assessment(s)) of user 10 and/or perform operation(s) to facilitate alteration (e.g., improvement) of user's 10 health, wellness, and/or well-being based on such assessment(s). As such, in certain embodiments described in the present disclosure, wearable device 100 can be capable of and/or configured to collect physiological sensor readings of user 10 and/or perform such assessment(s) and/or operation(s) using such readings.

However, in additional and/or alternative embodiments, wearable device 100 and/or another electronic and/or computing device that can be used to detect physiological information of user 10, can be in communication with external computing device 504. In these and/or other embodiments, external computing device 504 can be configured to use such physiological information of user 10 to perform such one or more health, wellness, and/or well-being assessments (e.g., physical, mental, emotional, behavioral, and/or sleep quality assessment(s)) of user 10 according to one or more embodiments described herein. In these and/or other embodiments, based at least in part on (e.g., in response to) performing such assessment(s), external computing device 504 can perform one or more operations described herein to facilitate alteration (e.g., improvement) of user's 10 health, wellness, and/or well-being (e.g., physical, mental, emotional, behavioral, and/or sleep quality).

Wearable device 100 according to example embodiments can be configured to collect one or more types of physiological and/or environmental data using embedded sensors and/or external devices, as described throughout the present disclosure, and communicate or relay such information over one or more networks 506 to other devices. This includes, in some embodiments, relaying information to devices capable of serving as Internet-accessible data sources, thus permitting the collected data to be viewed, for example, using a web browser or network-based application at, for instance, external computing device 504. For example, while user 10 is wearing wearable device 100, wearable device 100 can capture, calculate, and/or store environment data and/or user's 10 physiological data (e.g., heart rate, motion data, temperature, respiration, perspiration, EDA, stress data) using one or more environmental and/or physiological sensors. Wearable device 100 according to example embodiments can then transmit data representative of such environment data and/or user's 10 physiological data over network(s) 506 to an account on a web service, computer, mobile phone, and/or health station where the data can be stored, processed, and visualized by user 10 and/or another entity (e.g., a health care professional).

While wearable device 100 is shown in example embodiments of the present disclosure to have a display, it should be understood that, in some embodiments, wearable device 100 does not have any type of display unit. In some embodiments, wearable device 100 can have audio and/or visual feedback components such as, for instance, light-emitting diodes (LEDs), buzzers, speakers, and/or a display with limited functionality. Wearable device 100 according to example embodiments can be configured to be attached to user's 10 body or clothing. For example, in these or other embodiments, wearable device 100 can be configured as a wrist bracelet, watch, ring, electrode, finger-clip, toe-clip, chest-strap, ankle strap, and/or a device placed in a pocket. In additional or alternative embodiments, wearable device 100 can be embedded in something in contact with user 10 such as, for instance, clothing, a mat that can be positioned under user 10, a blanket, a pillow, and/or another accessory.

In one or more embodiments of the present disclosure, the communication between wearable device 100 and external computing device 504 can be facilitated by network(s) 506. In some embodiments, network(s) 506 can constitute and/or include, for instance, one or more of an ad hoc network, a peer-to-peer communication link, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the public switched telephone network (PSTN), a cellular telephone network, and/or any other type of network. In some embodiments, the communication between wearable device 100 and external computing device 504 can also be performed through a direct wired connection. In these or other embodiments, this direct-wired connection can be associated with any suitable or desirable communication protocol and/or physical connector such as, for instance, universal serial bus (USB), micro-USB, Wi-Fi, Bluetooth, FireWire, PCIe, or the like.

In example embodiments of the present disclosure, a variety of computing devices can be in communication with wearable device 100 to facilitate user's 10 health, wellness, and/or well-being assessment and/or alteration (e.g., improvement). Although external computing device 504 is depicted as a smartphone in the example embodiment illustrated in FIG. 5, it should be understood that the present disclosure is not so limiting. For instance, external computing device 504 according to example embodiments can constitute and/or include, for example, a smartphone with a display 508 as depicted in FIG. 5, a personal digital assistant (PDA), a mobile phone, a tablet, a personal computer, a laptop computer, a smart television, a video game console, a server, and/or another computing device that can be external to wearable device 100.

The networked relationship depicted in the example embodiment illustrated in FIG. 5 demonstrates how, in some embodiments, external computing device 504 can be implemented to perform one or more health, wellness, and/or well-being assessments (e.g., physical, mental, emotional, behavioral, and/or sleep quality assessment(s)) of user 10 and/or perform operation(s) to facilitate alteration (e.g., improvement) of user's 10 health, wellness, and/or well-being based on such assessment(s). For example, in one embodiment, user 10 can wear wearable device 100 that can be equipped as a bracelet with one or more physiological sensors but without a display. In this and/or another embodiment, while user 10 is wearing wearable device 100, wearable device 100 can capture, calculate, and/or store environment data and/or user's 10 physiological data (e.g., heart rate, motion data, temperature, respiration, perspiration, EDA, stress data) using one or more environmental and/or physiological sensors. Wearable device 100 according to example embodiments can then transmit data representative of such environment data and/or user's 10 physiological data over network(s) 506 to an account on a web service, computer, mobile phone, and/or health station where the data can be stored, processed, and visualized by user 10 and/or another entity (e.g., a health care professional). In some embodiments, wearable device 100 can periodically or continuously transmit such information to external computing device 504 over network(s) 506.

In additional and/or alternative embodiments, wearable device 100 can store the above-described collected physiological and/or environmental data and transmit this data to external computing device 504 in response to a trigger event such as, for instance, detection of user 10 being awake after a period of being asleep or detection of user 10 completing a defined activity (e.g., workout routine, exercise) after a period performing the defined activity. In some embodiments, wearable device 100 can transmit such data to external computing device 504 in response to detecting that a command has been performed by external computing device 504 such as, for instance, manual or automatic execution of an instruction to synchronize collected physiological and/or environmental data and perform one or more health, wellness, and/or well-being assessments (e.g., physical, mental, emotional, behavioral, and/or sleep quality assessment(s)) of user 10 as described herein.

In some embodiments, external computing device 504 can present (e.g., provide, render) a correlation or absence of correlation between a trigger event associated with physiological data of user 10 and at least one mood experienced by user 10 at a defined time associated with the trigger event. For instance, in these or other embodiments, external computing device 504 can generate an intelligent notification 510 that can include such correlation or absence of correlation and/or one or more health improvement recommendations (e.g., a suggestion to perform a defined activity to experience the at least one mood again) that, if and/or when implemented by user 10, can facilitate alteration (e.g., improvement) of user's 10 health, wellness, and/or well-being (e.g., physical, mental, emotional, behavioral, and/or sleep quality). In the example embodiment depicted in FIG. 5, external computing device 504 can render intelligent notification 510 having such correlation or absence of correlation and the health improvement recommendation(s) on display 508 such that user 10 and/or another entity (e.g., health care professional, mental health care professional, sleep therapy provider, doctor, caregiver) can view such information.

In some embodiments, external computing device 504 can: identify a correlation or absence of correlation between a trigger event associated with physiological data of user 10 and at least one mood experienced by user 10 at a defined time associated with the trigger event; determine one or more health improvement recommendations based on (e.g., in response to) identifying such a correlation or absence of correlation; generate intelligent notification 510 such that it includes the correlation or absence of correlation and the health improvement recommendation(s); and send this information back to wearable device 100 over network(s) 506 for presentation (e.g., via display 102) of such information to user 10 and/or another entity (e.g., health care professional, mental health care professional, sleep therapy provider, doctor, caregiver). Although not illustrated in the example embodiment depicted in FIG. 5, in some embodiments, wearable device 100 can: identify a correlation or absence of correlation between a trigger event associated with physiological data of user 10 and at least one mood experienced by user 10 at a defined time associated with the trigger event; determine one or more health improvement recommendations based on (e.g., in response to) identifying such a correlation or absence of correlation; generate intelligent notification 510 such that it includes the correlation or absence of correlation and the health improvement recommendation(s); and render this information on display 102 of wearable device 100.

In at least on embodiment, to identify a correlation or absence of correlation between a trigger event (e.g., including a defined activity as described herein) associated with physiological data of user 10 and at least one mood experienced by user 10 at a defined time associated with the trigger event, wearable device 100 and/or external computing device 504 can train an ML and/or AI model (e.g., a classifier) as described herein using the above-described annotated physiological dataset. In this embodiment, wearable device 100 and/or external computing device 504 can then implement the model to identify such a correlation or absence of correlation between such a trigger event (e.g., including a defined activity as described herein) and at least one mood experienced by user 10 at a defined time associated with the trigger event.

In at least one embodiment described herein, based at least in part (e.g., in response to) identify a correlation or absence of correlation between a trigger event associated with physiological data of user 10 and at least one mood experienced by user 10 at a defined time associated with the trigger event, wearable device 100 and/or external computing device 504 can, for example, implement and/or facilitate implementation of one or more wellness promoting features of wearable device 100 and/or external computing device 504. For instance, in this or another embodiment, wearable device 100 and/or external computing device 504 can implement and/or facilitate implementation of one or more wellness promoting features of wearable device 100 and/or external computing device 504 based at least in part on (e.g., in response to) detecting the trigger event associated with user's 10 physiological data.

In one embodiment of the present disclosure, wearable device 100 and/or external computing device 504 can implement (e.g., initiate, run, operate) one or more wellness promoting features that can be included with wearable device 100 and/or external computing device 504 such as, for instance, a wellness promoting audio feature (e.g., by playing wellness promoting music and/or sounds), a wellness promoting lighting feature (e.g., by initiating a “sleep mode” and/or “night mode” of wearable device 100 and/or external computing device 504 to dim one or more light sources of wearable device 100 and/or external computing device 504 such as a screen, display, or monitor), and/or another wellness promoting feature of wearable device 100 and/or external computing device 504.

For example, in this or another embodiment, wearable device 100 and/or external computing device 504 can cause an audio system of wearable device 100 and/or external computing device 504 to play wellness promoting music and/or sounds and/or cause a lighting system of wearable device 100 and/or external computing device 504 to initiate a “sleep mode” and/or “night mode” to dim one or more light sources of wearable device 100 and/or external computing device 504 such as a screen, display, or monitor.

In another embodiment of the present disclosure, wearable device 100 and/or external computing device 504 can facilitate implementation of one or more wellness promoting features of another computing device such as, for instance, a computing device of one or more smart systems 512. In this or another embodiment, smart system(s) 512 can constitute and/or include, but are not limited to, an audio system (e.g., a home audio system), a lighting system (e.g., a home lighting system), an HVAC system (e.g., a home HVAC system), an exercise system (e.g., an exercise machine), and/or another system that can be included in, coupled to, and/or operated by a computing device other than wearable device 100 and/or external computing device 504. For instance, in some embodiments, smart system(s) 512 can constitute and/or include a smart audio system, a smart lighting system, a smart HVAC system, and/or a smart exercise system (e.g., a smart exercise machine). In these or other embodiments, wearable device 100 and/or external computing device 504 can facilitate implementation of one or more wellness promoting features of smart system(s) 512 such as, for instance: a wellness promoting audio feature of a smart audio system; a wellness promoting lighting feature of a smart lighting system; a wellness promoting ambient temperature feature of a smart HVAC system; a wellness promoting exercise feature (e.g., a certain exercise mode or setting) of a smart exercise system; and/or another wellness promoting feature of smart system(s) 512.

In some embodiments described herein, wearable device 100 and/or external computing device 504 can send instructions to smart system(s) 512 that, when executed by such system(s) (e.g., via one or more processors), can cause the system(s) to perform operations to implement one or more wellness promoting features of such system(s). In one embodiment, wearable device 100 and/or external computing device 504 can send instructions to a smart audio system that, when executed by such a system (e.g., via one or more processors), can cause it to play wellness promoting music and/or sounds. In another embodiment, wearable device 100 and/or external computing device 504 can send instructions to a smart lighting system that, when executed by such a system (e.g., via one or more processors), can cause it to initiate a “sleep mode” and/or “night mode” to dim one or more light sources (e.g., light bulbs) of the smart lighting system. In another embodiment, wearable device 100 and/or external computing device 504 can send instructions to a smart HVAC system that, when executed by such a system (e.g., via one or more processors), can cause it to output air at a certain wellness promoting temperature (e.g., a certain temperature that can be defined by user 10). In one embodiment of the present disclosure, wearable device 100 and/or external computing device 504 can send instructions to a smart exercise system that, when executed by such a system (e.g., via one or more processors), can cause it to operate in a certain mode or setting and/or to provide a recommendation to the user to select such a mode or setting.

FIG. 6 illustrates a diagram of an example, non-limiting user assessment management system 600 according to one or more example embodiments of the present disclosure. User assessment management system 600 depicted in FIG. 6 illustrates an example, non-limiting networked relationship between one or more wearable devices 100a, 100b, 100c, one or more external computing devices 504a, 504b, 504c, and/or a server system 604 in accordance with one or more embodiments.

In the example embodiment depicted in FIG. 6, wearable devices 100a, 100b, 100c can each include the same characteristics, structure, components, attributes, and/or functionality as that of wearable device 100. In this embodiment, each wearable device 100a, 100b, 100c can be coupled to (e.g., worn by) a respective user 10a, 10b, 10c. In this embodiment, external computing devices 504a (e.g., a laptop computer), 504b (e.g., a smartphone), 504c (e.g., a personal computer) can each include the same characteristics, structure, components, attributes, and/or functionality as that of external computing device 504.

In some embodiments of the present disclosure, network(s) 506 can couple (e.g., communicatively) one or more of wearable devices 100a, 100b, 100c to server system 604 and/or one or more of external computing devices 504a, 504b, 504c. In some embodiments, one or more of external computing devices 504a, 504b, 504c and/or one or more of wearable devices 100a, 100b, 100c can be interconnected in a local area network (LAN) 602 or another type of communication interconnection that can be connected to (e.g., communicatively coupled to) network(s) 506. LAN 602 according to example embodiments can interconnect one or more of external computing devices 504a, 504b, 504c, as well as one or more of wearable devices 100a, 100b, 100c. In some embodiments, one or more of wearable devices 100a, 100b, 100c and/or one or more of external computing devices 504a, 504b, 504c can be connected to (e.g., communicatively coupled to) network(s) 506 and/or server system 604, indirectly, through LAN 602. In some embodiments, one or more of wearable devices 100a, 100b, 100c can be directly connected to (e.g., communicatively coupled to) network(s) 506 and/or indirectly connected to network(s) 506 through LAN 602. For instance, in the example embodiment depicted in FIG. 6, wearable device 100b can be connected to (e.g., communicatively coupled to) external computing device 504b (e.g., a smartphone) through, for example, a Bluetooth connection. In this embodiment, external computing device 504b can be connected to (e.g., communicatively coupled to) server system 604 through network(s) 506 and wearable device 100b can also be connected to (e.g., communicatively coupled to) server system 604 through network 506.

In the example embodiment depicted in FIG. 6, server system 604 can collect detected physiological and/or environmental sensor readings from one or more of wearable devices 100a, 100b, 100c. In some embodiments, server system 604 can also collect from one or more of wearable devices 100a, 100b, 100c and/or from one or more of external computing devices 504a, 504b, 504c, correlations or absences of correlation between trigger events respectively associated with physiological data of one or more users 10a, 10b, 10c and at least one mood respectively experienced by user(s) 10a, 10b, 10c at defined times respectively associated with the trigger events.

For example, in the embodiment depicted in FIG. 6, wearable device 100a is not associated with an external computing device, therefore wearable device 100a can transmit physiological data of user 10a to server system 604. In this embodiment, server system 604 can analyze the received data to identify a correlation or absence of correlation between a trigger event associated with physiological data of user 10a and at least one mood experienced by user 10a at a defined time associated with the trigger event. In this embodiment, server system 604 can transmit an intelligent notification (e.g., intelligent notification 510), the correlation or absence of correlation between the trigger event and the at least one mood experienced by user 10a, and/or one or more health improvement recommendations back to wearable device 100a.

As another example, in the embodiment depicted in FIG. 6, wearable device 100b can transmit physiological data of user 10b to server system 604 and external computing device 504a. In this embodiment, external computing device 504a can analyze the received data to identify a correlation or absence of correlation between a trigger event associated with physiological data of user 10b and at least one mood experienced by user 10b at a defined time associated with the trigger event. In this embodiment, server system 604 can use the received physiological data, correlation, or absence of correlation corresponding to user 10b to update a user profile for user 10b that can be stored in a profiles database 612 (e.g., a log) that can be stored on a memory 608 that can be included in, coupled to, and/or otherwise associated with server system 604.

In some embodiments, server system 604 can be implemented on one or more standalone data processing apparatuses or a distributed network of computers. In some embodiments, server system 604 can employ various virtual devices and/or services of third-party service providers (e.g., third-party cloud service providers) to provide the underlying computing resources and/or infrastructure resources of server system 604. In some embodiments, server system 604 can include, but is not limited to, a handheld computer, a tablet computer, a laptop computer, a desktop computer, or a combination of any two or more of these data processing devices or other data processing devices.

Server system 604 according to example embodiments can include one or more processors 606 (e.g., processing unit(s), denoted as “processor(s) 606” in FIG. 6) such as, for instance, one or more CPUs. In these or other embodiments, server system 604 can include one or more network interfaces 614 that can include, for example, an input/output (I/O) interface to external computing device 504a, 504b, and/or 504c and/or wearable devices 100a, 100b, and/or 100c. In some embodiments, server system 604 can include memory 608, and one or more communication buses for interconnecting these components.

Memory 608 according to example embodiments can include high-speed random-access memory such as, for instance, DRAM, SRAM, DDR RAM, or other random-access solid-state memory devices; and, optionally, can include non-volatile memory such as, for example, one or more magnetic disk storage devices, one or more optical disk storage devices, one or more flash memory devices, or one or more other non-volatile solid state storage devices. Memory 608 according to example embodiments, optionally, can include one or more storage devices that can be remotely located from processor(s) 606 (e.g., processing unit(s)). Memory 608 according to example embodiments, or alternatively the non-volatile memory within memory 608, can include a non-transitory computer readable storage medium. In some embodiments, memory 608, or the non-transitory computer readable storage medium of memory 608, can store one or more programs, modules, and data structures. In these embodiments, such programs, modules, and data structures can include, but not be limited to, one or more of an operating system that can include procedures for handling various basic system services and for performing hardware dependent tasks, a network communication module for connecting server system 604 to other computing devices (e.g., wearable device 100a, 100b, and/or 100c and/or external computing device 504a, 504b, and/ 504c) connected to network(s) 506 via network interface(s) 614 (e.g., wired or wireless).

Memory 608 according to example embodiments can include correlation or absence of correlation module 113 described above with reference to FIG. 4. As described above with reference to FIG. 4, in one embodiment, correlation or absence of correlation module 113 can constitute and/or include one or more of the ML and/or AI models described herein (e.g., a classifier) that can identify such a correlation or absence of correlation between the trigger event and the at least one mood experienced by user 10. In one embodiment, server system 604 can train such ML and/or AI model(s) as described herein using the above-described annotated physiological dataset. In one embodiment, server system 604 can implement (e.g., execute, run) correlation or absence of correlation module 113 and/or such ML and/or AI model(s) using collected physiological and/or environmental data of one or more users 10a, 10b, 10c (e.g., received from one or more wearable devices 100a, 100b, 100c or one or more external computing devices 504a, 504b, 504c) to identify correlations or absences of correlation between trigger events respectively associated with physiological data of one or more users 10a, 10b, 10c and at least one mood respectively experienced by user(s) 10a, 10b, 10c at defined times respectively associated with the trigger events.

Memory 608 according to example embodiments can also include profiles database 612 that can store user profiles for users 10a, 10b, 10c. In some embodiments, a respective user profile for a user can include, for instance: a user identifier (e.g., an account name or handle); login credentials (e.g., login credentials to user assessment management system 600); email address or preferred contact information; wearable device information (e.g., model number); demographic parameters for the user (e.g., age, gender, occupation); historical physiological data of the user; historical correlations or absences of correlation between trigger events and moods experienced by the user; and/or identified health, wellness, and/or well-being metrics and/or trends of the user (e.g., physical, mental, emotional, behavioral, sleep quality metrics and/or trends of the user).

In some embodiments, collected physiological information, as well as health, wellness, and/or well-being assessment information (e.g., physical, mental, emotional, behavioral, and/or sleep quality assessment information) of a plurality of users such as, for instance, users 10a, 10b, 10c of can provide for more robust population-normalized health, wellness, and/or well-being metrics and/or trends (e.g., physical, mental, emotional, behavioral, sleep quality metrics and/or trends). For example, in one embodiment, user 10a can be a 35 year old female veterinarian and user 10b can be a 34 year old female veterinarian. In this embodiment, each of their respective historical physiological data and health, wellness, and/or well-being assessment information (e.g., physical, mental, emotional, behavioral, and/or sleep quality assessment information) such as, for instance, their respective historical correlations or absences of correlation between trigger events and moods they respectively experienced, can be used in the determination of one or more population-normalized health, wellness, and/or well-being metrics and/or trends for each other, due to their closely aligned demographic characteristics.

In some embodiments, a user can opt in or opt out of providing health, wellness, and/or well-being assessment information (e.g., physical, mental, emotional, behavioral, and/or sleep quality assessment information) to a population-normalization determination for other users. In some embodiments, a user's health, wellness, and/or well-being assessment information (e.g., physical, mental, emotional, behavioral, and/or sleep quality assessment information) can be incorporated into population-normalized health, wellness, and/or well-being metric and/or trend information (e.g., physical, mental, emotional, behavioral, and/or sleep quality metric and/or trend information) used to determine that user's own values for one or more health, wellness, and/or well-being metrics and/or trends (e.g., physical, mental, emotional, behavioral, and/or sleep quality metrics and/or trends).

In at least one embodiment described herein, server system 604 can record, in profiles database 612, the health, wellness, and/or well-being assessment information (e.g., physical, mental, emotional, behavioral, and/or sleep quality assessment information) respectively corresponding to users 10a, 10b, 10c. In example embodiments of the present disclosure, with respect to each of such users 10a, 10b, 10c, such health, wellness, and/or well-being assessment information can include a plurality of correlations and a plurality of absences of correlation between a plurality of moods experienced by the user at a plurality of defined times respectively associated with a plurality of trigger events (e.g., including a plurality of defined activities performed by the user). In some embodiments, with respect to each of such users 10a, 10b, 10c, such health, wellness, and/or well-being assessment information can include the above-described annotated physiological dataset that can be used to train an ML and/or AI model described herein to identify such a plurality of correlations and plurality of absences of correlation.

In at least one embodiment, server system 604 can compare the health, wellness, and/or well-being assessment information (e.g., physical, mental, emotional, behavioral, and/or sleep quality assessment information) of a certain user 10a, 10b, or 10c with that of other users and further classify such a certain user in a defined correlation category or a defined absence of correlation category based at least in part on (e.g., in response to completing) such a comparison. For example, in one embodiment, server system 604 can classify such a certain user in a category including correlations between a certain exercise and a certain mood or a category including absences of correlation between such a certain exercise and certain mood.

In some embodiments, to perform the comparison and/or classification operations described above, server system 604 can use one or more of the ML and/or AI models described herein (e.g., a classifier). For example, in these embodiments, server system 604 can use such ML and/or AI model(s) to compare the plurality of correlations or the plurality of absences of correlation corresponding to a certain user (e.g., user 10a) to one or more other plurality of correlations or one or more other plurality of absences of correlation corresponding respectively to one or more other users (e.g., user 10b, 10c). In these embodiments, server system 604 can further use such ML and/or AI model(s) to classify such a certain user (e.g., user 10a) in a defined correlation category or a defined absence of correlation category based at least in part on (e.g., in response to completing) such a comparison.

In at least one embodiment of the present disclosure, based at least in part on (e.g., in response to) classifying a certain user (e.g., user 10a) in a defined correlation category or a defined absence of correlation category as described above, server system 604 can perform operations that can include, but are not limited to, for instance: informing (e.g., via an intelligent notification described above) the user and/or another computing device of such a classification; providing (e.g., via an intelligent notification described above) the user and/or another computing device with an explanation of such a classification of the user such the user understands why they are classified in such a category; suggesting one or more health improvement recommendations described herein and/or engage another computing device to make such recommendation(s) based at least in part on (e.g., using) such a classification of the user (e.g., recommendation that the user perform or avoid performing a certain activity to experience or avoid experiencing a certain mood, respectively); implementing one or more wellness promoting features described herein and/or engage another computing device to implement such feature(s) based at least in part on (e.g., using) such a classification of the user (e.g., playing certain music and/or sounds to encourage the user to perform a certain activity to experience a certain mood or to discourage the user from performing such an activity to avoid experiencing such a mood); and/or another operation according to one or more example embodiments of the present disclosure.

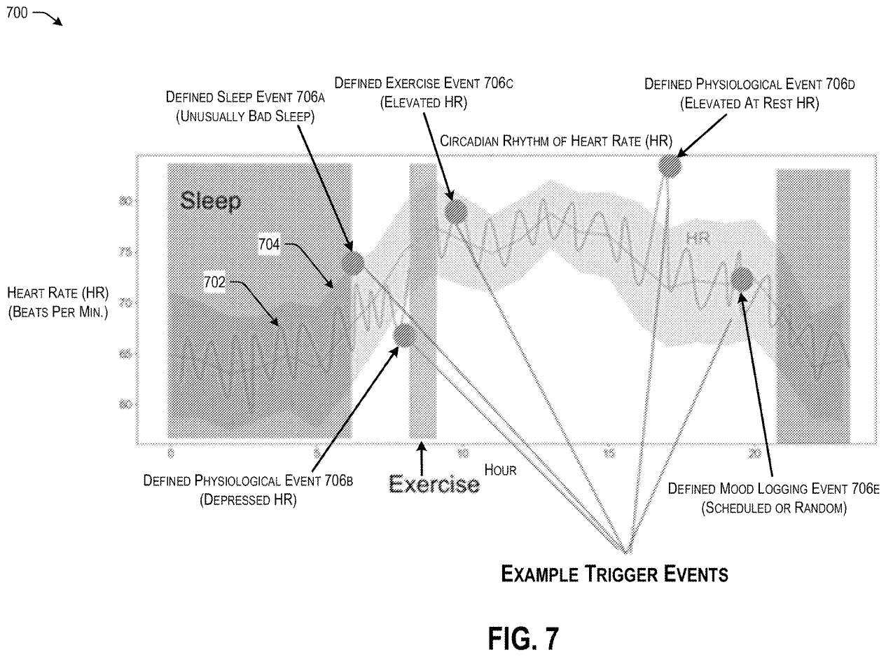

FIG. 7 illustrates an example, non-limiting physiological data graph 700 according to one or more example embodiments of the present disclosure. Physiological data graph 700 illustrated in the example embodiment depicted in FIG. 7 can constitute and/or include a plot 702 of a circadian rhythm of a user's heart rate (e.g., plotted as heart rate (HR) in beats per minute (min.) against hours over time) and a standard deviation 704 associated with such a circadian rhythm of the user's heart rate. Physiological data graph 700 illustrated in FIG. 7 can be used by a computing device (e.g., wearable device 100, 100a, 100b, 100c, external computing device 504, 504a, 504b, 504c, server system 604) according to example embodiments described herein to monitor such a circadian rhythm of the user's heart rate and/or to detect one or more trigger events associated with such physiological data of the user as described in example embodiments of the present disclosure.

As illustrated in the embodiment depicted in FIG. 7, plot 702 can include a defined sleep event 706a that can be indicative of and detected by the computing device as an unusually bad sleep session for the user. In this embodiment, the computing device can prompt the user to input how the user feels (e.g., by selecting one or more mood states on an interactive user interface) after the user awakes from the sleep session.

In the embodiment depicted in FIG. 7, plot 702 can further include a defined physiological event 706b that can be indicative of and detected by the computing device as a relatively depressed heart rate of the user while the user is awake. In this embodiment, the computing device can prompt the user to input how the user feels (e.g., by selecting one or more mood states on an interactive user interface) at the time the computing device detects defined physiological event 706b.

In the embodiment depicted in FIG. 7, plot 702 can further include a defined exercise event 706c that can be indicative of and detected by the computing device as a defined activity (e.g., yoga, jogging, briskly walking, swimming) that can be performed by the user. In this embodiment, the computing device can prompt the user to input how the user feels (e.g., by selecting one or more mood states on an interactive user interface) at a certain time (e.g., 1 minute, 10 minutes, 15 minutes) after completing defined exercise event 706c.

In the embodiment depicted in FIG. 7, plot 702 can further include a defined physiological event 706d that can be indicative of and detected by the computing device as a relatively elevated heart rate of the user while the user is at rest. In this embodiment, the computing device can prompt the user to input how the user feels (e.g., by selecting one or more mood states on an interactive user interface) at the time the computing device detects defined physiological event 706d.

In the embodiment depicted in FIG. 7, plot 702 can further include a defined mood logging event 706e that can be indicative of and detected by the computing device as a scheduled or random mood logging event. For example, in some embodiments, the computing device can allow for the user to define (e.g., input, select) one or more scheduled mood logging times when the computing device will prompt the user to input how the user feels (e.g., by selecting one or more mood states on an interactive user interface) at each of such scheduled mood logging times. In some embodiments, the computing device can randomly prompt the user to input how the user feels (e.g., by selecting one or more mood states on an interactive user interface) at the time the computing device randomly prompts the user. In some embodiments, the user can elect to randomly input how the user feels (e.g., by selecting one or more mood states on an interactive user interface) at some random time.

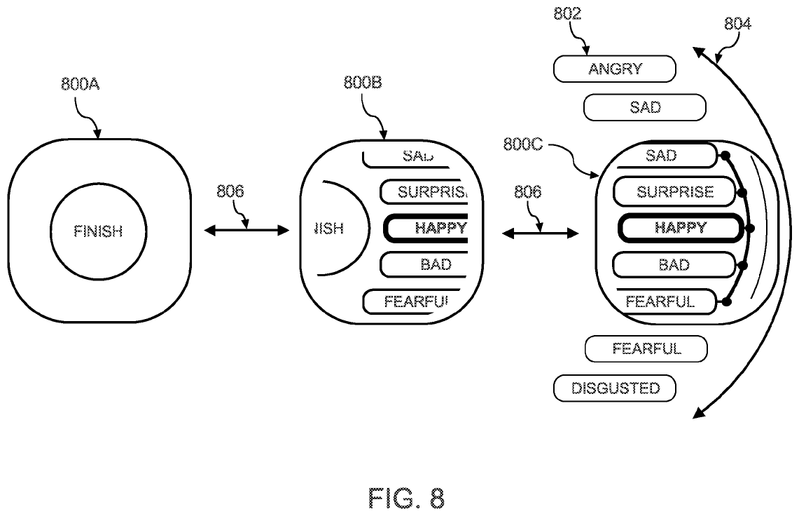

FIG. 8 illustrates example, non-limiting interactive user interfaces 800a, 800b, 800c according to one or more example embodiments of the present disclosure. In the embodiment depicted in FIG. 8, interactive user interface 800a, 800b, and/or 800c can constitute an interactive button wheel.

In the embodiment depicted in FIG. 8, interactive user interfaces 800a, 800b, 800c can each include one or more interactive user interface elements 802 (only a single interactive user interface element 802 is denoted in FIG. 8 for clarity). For example, in this embodiment, interactive user interface 800a can include a “FINISH” interactive user interface element 802. In this embodiment, each of interactive user interfaces 800b and 800c can include interactive user interface element(s) 802 such as, for instance, “ANGRY,” “SAD,” “SURPRISED,” “HAPPY,” “BAD,” “FEARFUL,” “DISGUSTED,” and/or another interactive user interface element 802. In this embodiment, each interactive user interface element 802 can constitute an interactive button that can be configured to receive input from a user by way of a touch (e.g., fingertip touch) by the user to indicate a selection by the user of the mood state labelled on the interactive button.

In at least one embodiment, based at least in part on (e.g., in response to) detecting a trigger event associated with a user's physiological data, a computing device according to example embodiments described herein (e.g., wearable device 100, 100a, 100b, 100c, external computing device 504, 504a, 504b, 504c, server system 604) can generate, configure, and/or render interactive user interfaces 800a, 800b, 800c on a display (e.g., monitor, screen, touch screen, capacitive touch screen, resistive touch screen) that can be coupled to the computing device. In this embodiment, the user can interact with interactive user interfaces 800a, 800b, 800c by moving between such interactive user interfaces 800a, 800b, 800c as indicated by arrows 806 and/or by cycling through interactive user interface elements 802 on interactive user interface 800c as indicated by arrow 804. In this embodiment, the user can select one or more interactive user interface elements 802 to input (e.g., log, record) at least one mood the user experienced at a defined time associated with the trigger event detected by the computing device.

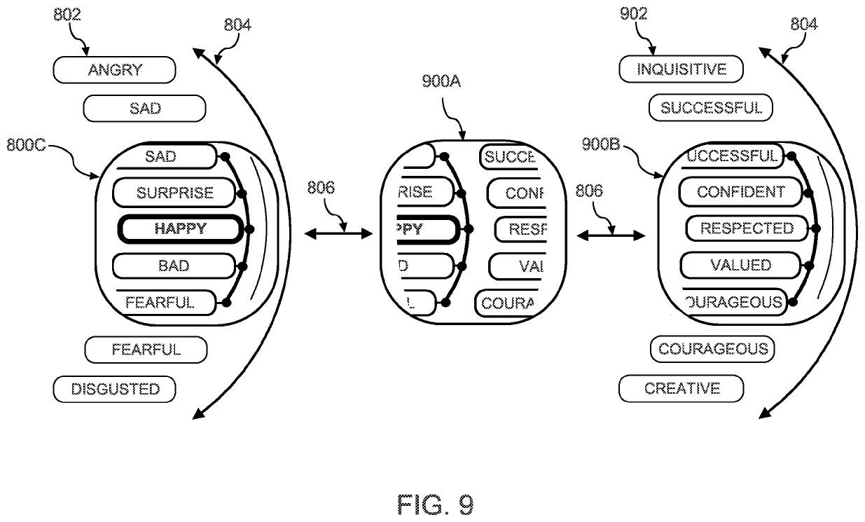

FIG. 9 illustrates example, non-limiting interactive user interfaces 800c, 900a, 900b according to one or more example embodiments of the present disclosure. In the embodiment depicted in FIG. 9, interactive user interfaces 900a, 900b can each constitute an example, non-limiting additional and/or alternative embodiment of interactive user interface 800c.

In some embodiments, with reference to the example embodiment described above and depicted in FIG. 8, a computing device according to example embodiments described herein (e.g., wearable device 100, 100a, 100b, 100c, external computing device 504, 504a, 504b, 504c, server system 604) can generate, configure, and/or render interactive user interface 800c as a primary interactive user interface (e.g., a primary interactive button wheel). In these embodiments, interactive user interface 800c can have one or more interactive user interface elements 802 (e.g., “ANGRY,” “SAD,” “SURPRISED,” “HAPPY,” “BAD,” “FEARFUL,” “DISGUSTED”) that can each constitute a primary interactive user interface element (e.g., primary interactive button) that can correspond to a primary mood state (e.g., general mood state).

With reference to the example embodiment depicted in FIG. 9, based at least in part on (e.g., in response to) a selection by the user of one or more interactive user interface elements 802 (e.g., primary mood state(s)) from interactive user interface 800c, the computing device can generate, configure, and/or render interactive user interface 900a and/or 900b as a secondary interactive user interface (e.g., a secondary interactive button wheel) that can constitute a sub-level of interactive user interface 800c. In this embodiment, the computing device can generate, configure, and/or render interactive user interfaces 900a, 900b such that each includes one or more interactive user interface elements 902 that can each constitute a secondary interactive user interface element (e.g., secondary interactive button) that can correspond to a secondary mood state. For example, in this embodiment, each secondary mood state can constitute a mood sub-state that can be relatively more specific and/or granular compared to the relatively more general primary mood state selected by the user from interactive user interface 800c.

In the embodiment depicted in FIG. 9, based at least in part on (e.g., in response to) a selection by the user of an interactive user interface element 802 corresponding to the “HAPPY” primary mood state from interactive user interface 800c, the computing device can generate, configure, and/or render interactive user interface 900a and/or 900b such that they include one or more interactive user interface elements 902 that can each correspond to a secondary mood state. For instance, in this embodiment, interactive user interface 900a and/or 900b can include interactive user interface element(s) 902 that can include, but not limited to, “INQUISITIVE,” “SUCCESSFUL,” “ENERGETIC,” “CONFIDENT,” “RESPECTED,” “VALUED,” “COURAGEOUS,” “CREATIVE,” and/or another interactive user interface element 902. In this embodiment, each interactive user interface element 902 can constitute an interactive button that can be configured to receive input from a user by way of a touch (e.g., fingertip touch) by the user to indicate a selection by the user of the secondary mood state labelled on the interactive button.

In the embodiment depicted in FIG. 9, based at least in part on (e.g., in response to) a selection by the user of an interactive user interface element 802 from interactive user interface 800c, the computing device can generate, configure, and/or render interactive user interface 900a and/or 900b on a display (e.g., monitor, screen, touch screen, capacitive touch screen, resistive touch screen) that can be coupled to the computing device. In this embodiment, the user can interact with interactive user interfaces 800c, 900a, 900b by moving between such interactive user interfaces 800c, 900a, 900b as indicated by arrows 806 and/or by cycling through interactive user interface elements 802 and/or 902 on interactive user interface 800c and/or 900b, respectively, as indicated by arrow 804. In this embodiment, the user can select one or more interactive user interface elements 802 and/or 902 to input (e.g., log, record) at least one primary mood and/or at least one secondary mood, respectively, that the user experienced at a defined time associated with a trigger event detected by the computing device.

### Example Methods

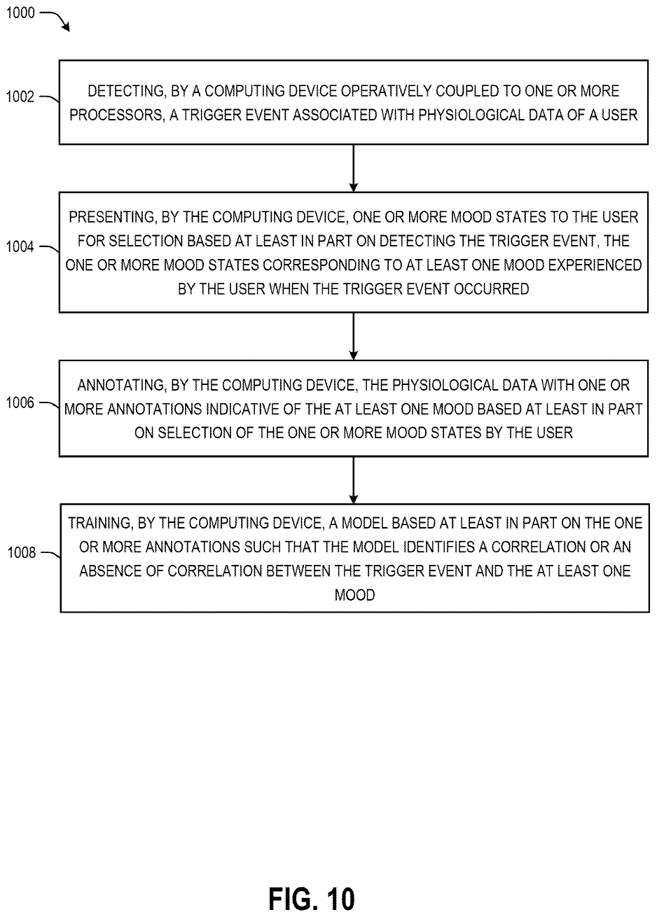

FIG. 10 illustrates a flow diagram of an example, non-limiting computer-implemented method 1000 according to one or more example embodiments of the present disclosure. Computer-implemented method 1000 can be implemented using, for instance, wearable device 100, 100a, 100b, and/or 100c, external computing device 504, 504a, 504b, and/or 504c, and/or server system 604 described above with reference to the example embodiments depicted in FIGS. 1, 2, 3, 4, 5, and 6.

The example embodiment illustrated in FIG. 10 depicts operations performed in a particular order for purposes of illustration and discussion. Those of ordinary skill in the art, using the disclosures provided herein, will understand that various operations or steps of computer-implemented method 1000 or any of the other methods disclosed herein can be adapted, modified, rearranged, performed simultaneously, include operations not illustrated, and/or modified in various ways without deviating from the scope of the present disclosure.

At 1002, computer-implemented method 1000 can include detecting, by a computing device (e.g., wearable device 100, 100a, 100b, and/or 100c, external computing device 504, 504a, 504b, and/or 504c, and/or server system 604) operatively coupled to one or more processors (e.g., processor(s) 181, processor(s) 606), a trigger event associated with physiological data of a user.

At 1004, computer-implemented method 1000 can include presenting, by the computing device, one or more mood states to the user for selection based at least in part on detecting the trigger event, the one or more mood states corresponding to at least one mood experienced by the user at a defined time associated with the trigger event.

At 1006, computer-implemented method 1000 can include annotating, by the computing device, the physiological data with one or more annotations indicative of the at least one mood based at least in part on selection of the one or more mood states by the user.

At 1008, computer-implemented method 1000 can include training, by the computing device, a model based at least in part on the one or more annotations such that the model identifies a correlation or an absence of correlation between the trigger event and the at least one mood.

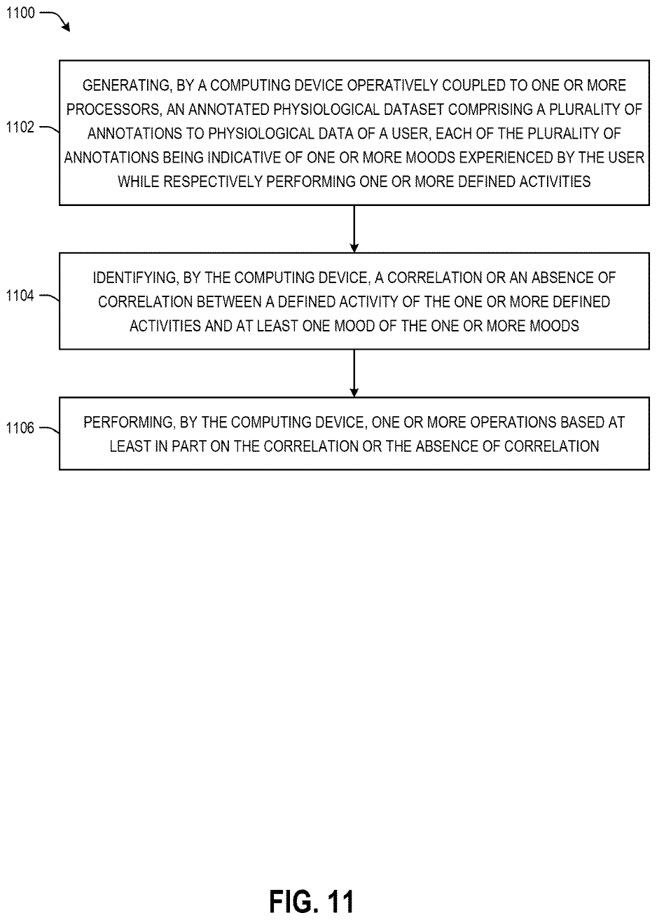

FIG. 11 illustrates a flow diagram of an example, non-limiting computer-implemented method 1100 according to one or more example embodiments of the present disclosure. Computer-implemented method 1100 can be implemented using, for instance, wearable device 100, 100a, 100b, and/or 100c, external computing device 504, 504a, 504b, and/or 504c, and/or server system 604 described above with reference to the example embodiments depicted in FIGS. 1, 2, 3, 4, 5, and 6.

The example embodiment illustrated in FIG. 11 depicts operations performed in a particular order for purposes of illustration and discussion. Those of ordinary skill in the art, using the disclosures provided herein, will understand that various operations or steps of computer-implemented method 1100 or any of the other methods disclosed herein can be adapted, modified, rearranged, performed simultaneously, include operations not illustrated, and/or modified in various ways without deviating from the scope of the present disclosure.

At 1102, computer-implemented method 1100 can include generating, by a computing device (e.g., wearable device 100, 100a, 100b, and/or 100c, external computing device 504, 504a, 504b, and/or 504c, and/or server system 604) operatively coupled to one or more processors (e.g., processor(s) 181, processor(s) 606), an annotated physiological dataset including a plurality of annotations to physiological data of a user, each of the plurality of annotations being indicative of one or more moods experienced by the user at each of one or more defined times respectively associated with one or more defined activities performed by the user.

At 1104, computer-implemented method 1100 can include identifying, by the computing device, a correlation or an absence of correlation between a defined activity of the one or more defined activities and at least one mood of the one or more moods.

At 1106, computer-implemented method 1100 can include performing, by the computing device, one or more operations based at least in part on the correlation or the absence of correlation.

### Additional Disclosure

Further to the descriptions above, a user may be provided with controls allowing the user to make an election as to both if and when systems, programs, or features described herein may enable collection of user information (e.g., information about a user's social network, social actions, or activities, profession, a user's preferences, or a user's current location), and if the user is sent content or communications from a server. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user. To that end, any information collected as described herein relating to the user will be kept private and confidential and will not be improperly used or published.

The technology discussed herein makes reference to servers, databases, software applications, and other computer-based systems, as well as actions performed by, and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure covers such alterations, variations, and equivalents.

What is claimed is:

1. A computing device, comprising:
one or more processors; and
one or more computer-readable media that collectively store instructions that, when executed by the one or more processors, cause the computing device to perform operations, the operations comprising:
detecting a trigger event associated with physiological data of a user;
presenting one or more mood states to the user for selection based at least in part on detecting the trigger event, the one or more mood states corresponding to at least one mood experienced by the user at a defined time associated with the trigger event;
annotating the physiological data with one or more annotations indicative of the at least one mood based at least in part on selection of the one or more mood states by the user; and
training a model based at least in part on the one or more annotations such that the model identifies a correlation or an absence of correlation between the trigger event and the at least one mood.

2. The computing device of claim 1, wherein the trigger event comprises at least one of a defined physiological event, a defined activity event, a defined sleep event, a defined behavioral event, a defined exercise event, or a defined mood logging event.

3. The computing device of claim 1, wherein presenting the one or more mood states to the user for selection based at least in part on detecting the trigger event comprises:
rendering an interactive user interface on a display coupled to the computing device, the interactive user interface comprising one or more interactive user interface elements that respectively correspond to the one or more mood states,
wherein each of the one or more interactive user interface elements is configured to receive input that is indicative of a selection of a mood state of the one or more mood states.

4. The computing device of claim 1, wherein the one or more annotations each comprise at least one of one or more metadata or tags indicative of the at least one mood, and wherein annotating the physiological data with the one or more annotations indicative of the at least one mood based at least in part on selection of the one or more mood states by the user comprises at least one of:
annotating one of one or more physiological data values of the physiological data with the one or more annotations indicative of the at least one mood based at least in part on selection of the one or more mood states by the user; or
annotating a vector representation of the physiological data with the one or more annotations indicative of the at least one mood based at least in part on selection of the one or more mood states by the user.

5. The computing device of claim 1, wherein the operations further comprise:
annotating the physiological data with a plurality of annotations that are each indicative of one or more moods experienced by the user at each of a plurality of defined times respectively associated with a plurality of trigger events; and
generating an annotated physiological dataset comprising the plurality of annotations,
wherein the plurality of annotations comprise the one or more annotations, the one or more moods comprise the at least one mood, the plurality of defined times comprise the defined time, and the plurality of trigger events comprise the trigger event.

6. The computing device of claim 5, wherein training the model based at least in part on the one or more annotations such that the model identifies the correlation or the absence of correlation between the trigger event and the at least one mood comprises:
training the model using the annotated physiological dataset such that the model identifies the correlation or the absence of correlation between the trigger event and the at least one mood.

7. The computing device of claim 1, wherein the operations further comprise:
generating an intelligent notification comprising the correlation or the absence of correlation; and
providing the intelligent notification to at least one of the user or a second computing device.

8. The computing device of claim 1, wherein the operations further comprise:
generating an intelligent notification comprising a recommendation that the user perform a defined health improvement activity to experience the at least one mood or to avoid experiencing the at least one mood; and
providing the intelligent notification to at least one of the user or a second computing device.

9. A computer-implemented method, comprising:
detecting, by a computing device operatively coupled to one or more processors, a trigger event associated with physiological data of a user;
presenting, by the computing device, one or more mood states to the user for selection based at least in part on detecting the trigger event, the one or more mood states corresponding to at least one mood experienced by the user at a defined time associated with the trigger event;
annotating, by the computing device, the physiological data with one or more annotations indicative of the at least one mood based at least in part on selection of the one or more mood states by the user; and
training, by the computing device, a model based at least in part on the one or more annotations such that the model identifies a correlation or an absence of correlation between the trigger event and the at least one mood.

10. The computer-implemented method of claim 9, wherein the trigger event comprises at least one of a defined physiological event, a defined activity event, a defined sleep event, a defined behavioral event, a defined exercise event, or a defined mood logging event.

11. The computer-implemented method of claim 9, wherein presenting, by the computing device, the one or more mood states to the user for selection based at least in part on detecting the trigger event comprises:
rendering, by the computing device, an interactive user interface on a display coupled to the computing device, the interactive user interface comprising one or more interactive user interface elements that respectively correspond to the one or more mood states,
wherein each of the one or more interactive user interface elements is configured to receive input that is indicative of a selection of a mood state of the one or more mood states.

12. The computer-implemented method of claim 9, wherein annotating, by the computing device, the physiological data with the one or more annotations indicative of the at least one mood based at least in part on selection of the one or more mood states by the user comprises at least one of:
annotating, by the computing device, one of one or more physiological data values of the physiological data with the one or more annotations indicative of the at least one mood based at least in part on selection of the one or more mood states by the user; or
annotating, by the computing device, a vector representation of the physiological data with the one or more annotations indicative of the at least one mood based at least in part on selection of the one or more mood states by the user.

13. The computer-implemented method of claim 9, further comprising:
annotating, by the computing device, the physiological data with a plurality of annotations that are each indicative of one or more moods experienced by the user at each of a plurality of defined times respectively associated with a plurality of trigger events; and
generating, by the computing device, an annotated physiological dataset comprising the plurality of annotations,
wherein the plurality of annotations comprise the one or more annotations, the one or more moods comprise the at least one mood, the plurality of defined times comprise the defined time, and the plurality of trigger events comprise the trigger event.

14. The computer-implemented method of claim 13, wherein training, by the computing device, the model based at least in part on the one or more annotations such that the model identifies the correlation or the absence of correlation between the trigger event and the at least one mood comprises:
training, by the computing device, the model using the annotated physiological dataset such that the model identifies the correlation or the absence of correlation between the trigger event and the at least one mood.

15. A computing device, comprising:
one or more processors; and
one or more computer-readable media that collectively store instructions that, when executed by the one or more processors, cause the computing device to perform operations, the operations comprising:
generating an annotated physiological dataset comprising a plurality of annotations to physiological data of a user, each of the plurality of annotations being indicative of one or more moods experienced by the user at each of one or more defined times respectively associated with one or more defined activities performed by the user;
identifying a correlation or an absence of correlation between a defined activity of the one or more defined activities and at least one mood of the one or more moods; and
performing one or more operations based at least in part on the correlation or the absence of correlation.

16. The computing device of claim 15, wherein performing the one or more operations based at least in part on the correlation or the absence of correlation comprises:
generating an intelligent notification comprising the correlation or the absence of correlation; and
providing the intelligent notification to at least one of the user or a second computing device.

17. The computing device of claim 15, wherein performing the one or more operations based at least in part on the correlation or the absence of correlation comprises:
generating an intelligent notification comprising a recommendation that the user perform the defined activity to experience the at least one mood or avoid performing the defined activity to avoid experiencing the at least one mood; and
providing the intelligent notification to at least one of the user or a second computing device.

18. The computing device of claim 15, wherein performing the one or more operations based at least in part on the correlation or the absence of correlation comprises:
implementing one or more wellness promoting features of at least one of the computing devices or a second computing device based at least in part on the correlation or the absence of correlation.

19. The computing device of claim 18, wherein the one or more wellness promoting feature is implemented when a trigger event is detected and if a correlation between the trigger event and one or more mood had been identified.

20. The computing device of claim 19, wherein implementing the wellness promoting feature comprises activating a feature of a unit of the computing device and/or at least one external device.

21. The computing device of claim 20, wherein the external device comprises at least one of an exercise system, an audio/video system, a lighting system and a HVAC.

22. The computing device of claim 15, wherein performing the one or more operations based at least in part on the correlation or the absence of correlation comprises:
recording, in a database, a plurality of correlations and a plurality of absences of correlation between a plurality of moods experienced by the user at a plurality of defined times respectively associated with a plurality of defined activities performed by the user,
wherein the plurality of correlations comprise the correlation and the plurality of absences of correlation comprise the absence of correlation.

23. The computing device of claim 19, wherein performing the one or more operations based at least in part on the correlation or the absence of correlation comprises:
classifying the user in a defined correlation category or a defined absence of correlation category based at least in part on comparison of at least one of the plurality of correlations or the plurality of absences of correlation corresponding to the user to at least one of one or more second plurality of correlations or one or more second plurality of absences of correlation corresponding respectively to one or more second users.
